(19) United States
(12) Patent Application Publication
(10) Pub. No.: US 2022/0211029 A1
(43) Pub. Date: Jul. 7, 2022

(54) FREEZE-DRIED PERIPHERAL BLOOD MONONUCLEAR CELL COMPOSITIONS AND METHODS

(71) Applicant: Cellphire, Inc., Rockville, MD (US)

(72) Inventors: Keith Andrew Moskowitz, Westfield, IN (US); Daniel Allen Sheik, Rockville, MD (US); Michael Alexander Matthews, Arlington, VA (US); Stephen Edward Amos, Buckeystown, MD (US)

(73) Assignee: Cellphire, Inc., Rockville, MD (US)

(21) Appl. No.: 17/523,722

(22) Filed: Nov. 10, 2021

Related U.S. Application Data
(60) Provisional application No. 63/111,975, filed on Nov. 10, 2020.

(51) Int. Cl. A01N 1/02; C12N 5/078

(52) U.S. Cl. CPC A01N 1/0221; C12N 2501/998; C12N 2500/34; C12N 5/0634

(57) ABSTRACT

Provided herein are methods of preserving PBMCs, including lyophilizing PBMCs, and compositions thereof. In some embodiments, such compositions include freeze-dried or rehydrated PBMCs in an aqueous mixture comprising a cryoprotectant, a lyoprotectant, and a buffer. In some embodiments, such compositions further include a PBMC cell culture media. Furthermore, provided herein are methods for preparing freeze-dried PBMCs and for preparing rehydrated PBMCs, as well as methods for administering freeze-dried PBMCs.

### CROSS-REFERENCE TO RELATED APPLICATIONS

This application claims the benefit of U.S. Provisional Application No. 63/111,975, filed Nov. 10, 2020, which is incorporated by reference herein in its entirety.

### TECHNICAL FIELD

Provided herein are methods for preserving nucleated cells, such as peripheral blood mononuclear cells (PBMCs), including lyophilized PBMCs, such as, for example, methods that preserve cell marker integrity and viability. Also provided herein are compositions of preserved nucleated cells, such as PBMCs, including lyophilized PBMCs.

### BACKGROUND

Blood is a complex mixture of numerous components. In general, blood can be described as comprising four main parts: red blood cells, white blood cells, platelets, and plasma. The first three are cellular or cell-like components, whereas the fourth (plasma) is a liquid component comprising a wide and variable mixture of salts, proteins, and other factors necessary for numerous bodily functions. The components of blood can be separated from each other by various methods. In general, differential centrifugation is most commonly used currently to separate the different components of blood based on size and, in some applications, density.

Peripheral blood is fluid that travels through your heart, arteries, capillaries and veins. Typically, peripheral blood mononuclear cells (PBMCs), which include lymphocytes, such as for example, T cells, B cells, Natural Killer cells, and monocytes, whereas erythrocytes and platelets have no nuclei. In humans, lymphocytes make up the majority of the PBMC population, followed by monocytes, and only a small percentage of dendritic cells.

PBMCs have been increasingly important as tools for cell-based therapeutics. For example, CAR-T therapy and therapeutic NK cells are important cell types for immune-oncology. Thus, methods for preserving such cells and the availability of preserved T cells and NK cells, for example, would provide important tools for treating cancer.

### SUMMARY

Provided herein in certain aspects and embodiments are compositions that include preserved nucleated cells (e.g. PBMCs), freeze-dried nucleated cells (e.g. PBMCs) or rehydrated nucleated cells (e.g. PBMCs). Such embodiments/aspects typically include freeze-dried PBMCs or rehydrated PBMCs in a composition that includes components that were included in a mixture in which the PBMCs were suspended before being freeze-dried. Freeze-dried PBMCs are in solid form, for example as a powder. Rehydrated PBMCs are in liquid form. Effective components, combinations of such components, and concentration ranges for such mixtures, and the resulting PBMC, freeze-dried PBMC, and rehydrated PBMC compositions are provided herein. Furthermore, numerous aspects and embodiments are provided herein as non-limiting examples of such compositions. In illustrative embodiments, such compositions include freeze-dried or rehydrated PBMCs suspended in (for rehydrated compositions), or dried with the components of (for freeze-dried compositions), an aqueous mixture comprising a cryoprotectant, a lyoprotectant, and a buffer. Non-limiting examples of each type of component, are provided herein, as are numerous non-limiting combinations of these components. In certain illustrative embodiments, the compositions comprise a buffer, trehalose, polysucrose, and either or both DMSO and albumin. Such compositions can further include one or more additional sugars and an alcohol, such as ethanol. In further examples of such illustrative embodiments, as non-limiting examples where albumin is included, the composition further includes the components of a PBMC cell culture media at effective concentrations for culturing PBMCs. It will be understood that the buffer in such compositions can be the buffer provided by the PBMC cell culture media.

In some embodiments and aspects provided herein are processes/methods for preparing freeze-dried (e.g., lyophilized) PBMCs and for preparing rehydrated PBMCs that had been freeze-dried. The processes/methods typically include the following steps:

A) incubating PBMCs in a liquid, typically an aqueous solution that includes a cryoprotectant, which in illustrative embodiments is trehalose and/or DMSO, and a lyoprotectant, that in illustrative embodiments is polysucrose, DMSO, and/or albumin; and
B) lyophilizing the liquid to form freeze-dried PBMCs.

Such methods are also provided herein as methods for preserving or enhancing metabolic activity or methods of preserving viability of a population of nucleated cells (e.g. PBMCs).

Some aspects provided herein, are methods for administering rehydrated, preserved nucleated cells (e.g. rehydrated PBMCs) to a subject.

Further details regarding aspects and embodiments of the present disclosure are provided throughout this patent application. Sections and section headers are for ease of reading and are not intended to limit combinations of disclosure, such as methods, compositions, and kits or functional elements therein across sections.

### BRIEF DESCRIPTION OF THE DRAWINGS

FIG. 1 shows a photograph of an MTT assay of fresh PBMCs under various conditions.

FIGS. 2A-C shows a photograph of an MTT assay of lyophilized PBMCs under various conditions.

FIG. 3 is a graph showing % maximum (“Max) activity comparing fresh PBMCs and lyophilized PBMCs.

FIG. 4 is a graph showing the optical density (OD) of fresh PBMCs after MTT assay under various conditions.

### DEFINITIONS

It is to be understood that the terminology used herein is for the purpose of describing particular embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9, 1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this specification by the term “about” when used in conjunction with a range. Furthermore, the term “about” when used to modify a specific recited value, is intended to cover+/−5% of the recited value. Thus, as a non-limiting example, “about 100,” is intended to mean 95 to 105.

Unless defined otherwise, all technical and scientific terms used herein have the same meaning as commonly understood by one of ordinary skill in the art to which the term belongs. Although any methods and materials similar or equivalent to those described herein can be used in the practice or testing of the present invention, exemplary methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “a PBMC” includes a plurality of such PBMCs. Furthermore, the use of terms that can be described using equivalent terms include the use of those equivalent terms. Thus, for example, the use of the term “subject” is to be understood to include the terms “patient”, “individual” and other terms used in the art to indicate one who is subject to a treatment.

As used herein the term “peripheral blood mononuclear cell(s)” or “PBMC(s)” are any peripheral blood cell(s) having a single nucleus. In contrast, other blood cells such as platelets and erythrocytes lack a nucleus. In some embodiments, PBMCs are lymphocytes, such as for example, T-cells, B-cells, and natural killer cells. In some embodiments, PBMCs are monocytes. In some embodiments, such monocytes are or comprise dendritic cells and/or macrophages. In some embodiments, PBMCs are dendritic cells. In some embodiments, the methods and compositions described herein encompass a mixture of PBMCs, such as for example, two or more types of PBMCs. In some embodiments, the PBMCs are all of the same type, or substantially the same type. For example, PBMCs can be all or substantially all B-cells, all or substantially all T-cells, all or substantially all lymphocytes (in any proportion of B-cells and T-cells), all or substantially all monocytes, etc.

### DETAILED DESCRIPTION

Reference will now be made in detail to various exemplary aspects and embodiments of the methods and compositions described herein. It is to be understood that the following detailed description is provided to assist the reader in understanding certain features and embodiments of the methods and compositions described herein, and that the following detailed description is not to be understood as limiting the methods and compositions described herein to the particular details specifically discussed.

Provided herein in certain aspects, are preserved nucleated cells, such as stem cells, including in illustrative embodiments preserved peripheral blood mononuclear cells (PBMCs), which in further illustrative embodiments are freeze-dried nucleated cells, stem cells, and PBMCs. Furthermore, provided herein are processes/methods for preserving such nucleated cells. Such preserved nucleated cells (e.g. PBMCs) provide important therapeutic tools that can be stored for long-periods of time, and are readily available to treat disorders and disease, such as cancer, in a subject. Thus, compositions provided herein provide important therapeutic tools to overcome of the most challenging diseases/disorders.

Freeze-Dried/Rehydrated PBMC Compositions

Accordingly, provided herein in certain aspects and embodiments are compositions that include freeze-dried PBMCs or rehydrated PBMCs. Such embodiments/aspects typically include freeze-dried PBMCs or rehydrated PBMCs in a composition that includes components that were included in a mixture in which the PBMCs were suspended before being freeze-dried. Freeze-dried PBMCs are in solid form, for example as a powder. Rehydrated PBMCs are in liquid form. Effective components, combinations of such components, and concentration ranges for such mixtures, and the resulting PBMC, freeze-dried PBMC, and rehydrated PBMC compositions are provided herein. Furthermore, numerous aspects and embodiments are provided herein as non-limiting examples of such compositions. In illustrative embodiments, such compositions include freeze-dried or rehydrated PBMCs suspended in (for rehydrated compositions), or dried with the components of (for freeze-dried compositions), an aqueous mixture comprising a cryoprotectant, a lyoprotectant, and a buffer. Non-limiting examples of each type of component, are provided herein, as are numerous non-limiting combinations of these components. In certain illustrative embodiments, the compositions comprise a buffer, trehalose, polysucrose, and either or both DMSO and albumin. Such compositions can further include one or more additional sugars and an alcohol, such as ethanol. In further examples of such illustrative embodiments, as non-limiting examples where albumin is included, the composition further includes the components of a PBMC cell culture media at effective concentrations for culturing PBMCs. It will be understood that the buffer in such compositions can be the buffer provided by the PBMC cell culture media.

With respect to rehydrated PBMC compositions, as indicated above, typically the components of the rehydrated composition are those in which the PBMCs were freeze-dried. Furthermore, in illustrative embodiments, freeze-dried PBMCs are rehydrated to about or exactly the same volume in which they were freeze-dried. Thus, the freeze-dried PBMCS and other components of the rehydrated compositions in illustrative embodiments, are present at about or the same concentrations in which they were present in the liquid PBMC composition that was freeze-dried before being rehydrated. Many different aqueous liquids/compositions can be used to rehydrate the freeze-dried PBMCs as discussed herein with respect to processes for preparing rehydrated PBMCs. In illustrative embodiments, water is used. However, in other embodiments a saline solution is used, a buffer is used, a water/buffer plasma mixture is used, or a PBMC cell culture media is used to rehydrate the freeze-dried PBMC composition. Thus, since certain illustrative embodiments include PBMCs rehydrated in PBMC cell culture media before freeze-drying and/or after rehydration, in certain illustrative embodiments rehydrated PBMCs are suspended in a liquid/mixture that includes a PBMC cell culture media that includes components of same at effective concentrations for supporting PBMC culturing. In further illustrative embodiments, PBMC cell culture media components are at the target, typically the published and/or source recommended concentrations for such PBMC cell culture media.

Such compositions include a population of PBMCs. In some embodiments, such population can include between 1×10⁵, 1×10⁶, or 1×10⁷, on the low end of the range, and 1×10⁸, 1×10⁹, 1×10¹⁰, 1×10¹¹, 1×10¹² freeze-dried PBMCs on the high end of the range. As disclosed herein, a certain percentage of the PBMCs in the freeze-dried population of PBMCs when tested after rehydration (i.e. when rehydrated), and in a rehydrated population of PBMCs, remain viable and a percentage remain metabolically active. In some embodiments, a PBMC composition comprising freeze-dried PBMCs in the form of a solid, or a PBMC composition comprising freeze-dried PBMCs, when rehydrated, as described herein, between 1% and 25%, for example, between 1 and 20%, 5 and 20%, 1 and 10%, 5 and 10%, 5 and 8%, or between 1 and 8% of the freeze-dried PBMCs are metabolically active and/or viable.

In some embodiments, a PBMC composition comprising DMSO, such that the PBMC composition in the form of a liquid, or the PBMC composition in the form of a solid, when rehydrated, between 10 and 25%, 12 and 25%, 15 and 25%, 17 and 25%, 10 and 20%, 12 and 20%, 15 and 20%, or 17 and 20%, freeze-dried PBMCs are viable.

In some embodiments, a PBMC composition as described herein, does not comprise DMSO. In some embodiments, a process for preparing a PBMC composition, or a PBMC composition prepared by a process for preparing a PBMC composition as described herein, the process at any step does not comprise contacting any of the components during the process with DMSO. In some embodiments, a PBMC composition does not comprise DMSO, and in such a composition in the liquid form or when the composition in the form of solid is rehydrated, between between 1 and 10%, 1 and 9%, 1 and 8%, 1 and 7%, 5 and 10%, 5 and 9%, 5 and 8%, or 5 and 7% of the freeze-dried PBMCs are metabolically active and/or viable.

In some embodiments, in a PBMC composition in the form of solid comprising freeze-dried PBMCs as described herein, the PBMCs are never suspended in a liquid comprising DMSO during the method of preparing such a PBMC composition. In some embodiments, in a PBMC composition in the form of liquid comprising rehydrated freeze-dried PBMCs as described herein, the PBMCs are never suspended in a liquid comprising DMSO during the method of preparing such a PBMC composition. It is to be understood that in certain embodiments, a PBMC composition as described herein when prepared by a process as described herein does not include a step wherein the PBMCs and/or freeze-dried PBMCs are contacted with DMSO. In some embodiments, not limiting a PBMC composition as described herein by a process, such a PBMC composition does not comprise DMSO as one of its constituents.

In some embodiments, herein PBMC compositions, whether freeze-dried or rehydrated, are in one or a plurality of vessels or containers, such as vials or tubes. Each of the container that comprises a PBMC composition in the form of a solid can be rehydrated to a target volume before clinical administration. Thus, certain embodiments herein include any of the PBMC compositions herein in a vessel, container, vial, and/or tube. The PBMC composition in the form of a solid as described herein can be contained in containers/vials, which further can be packed into a plurality of containers for shipping to a customer, which can be part of a commercialization process to fulfill an order for such PBMC composition. The containers, in certain embodiments, are 5 ml vials, 10 ml vials, 20 ml vials, 25 ml vials, 30 ml vials, 40 ml vials, 50 ml vials, 60 ml vials, 75 ml vials, 100 ml vials, 125 ml vials, 150 ml vials, 200 ml vials, or 250 ml vials. In some embodiments, the volume of the containers in a plurality of containers (e.g. vials or tubes), which for example can be all from one lot, or from more than one lot (e.g. 2, 3, 4, 5, 6, 7, 8, 9 or 10 lots), can vary from one or more than one size between 10-100 ml. Typically, the volume of the vial/container in embodiments where the freeze-dried PBMCs is a freeze-dried solid/powder, is 1× the volume of, or 1.10, 1.25, 1.5, 2, 2.5, 3, 4 or 5 times the volume of a composition that was filled in the vial before lyophilization, and/or the volume in which the powder in the vials will be rehydrated, which is an illustrative embodiment. Thus, the maximum volume of such vials can be the same or more than the volume of the composition that was filled inside prior to lyophilization or the volume in which the PBMC composition in the form of a powder can be rehydrated. For example, in one non-limiting embodiment, a vial with a maximum capacity of 100 ml, can be used to fill 10 ml of a composition that includes PBMCs for lyophilization. In certain embodiments, the capacity of a vial in which a PBMC composition that includes PBMCs is lyophilized, is 1-2.5 times and in other embodiments, 1-2 times, 1-3 times, 1-4 times, 1-5 times, and in certain illustrative embodiments, 1.1 to 2 times or 1.25 to 2 times the volume of a composition that is lyophilized therein. In some embodiments, a batch/lot can have 10-500 vials, 25-450 vials, 50-350 vials, 100-300 vials, or 150-250 vials. In some embodiments, a batch/lot can have 10, 20, 30, 40, 50, 60, 70, 80, 90, 100, 110, 120, 130, 140, 150, 160, 170, 180, 190, 200, 250, 300, 350, 400, 450, or 500 vials. In some embodiments, the number of vials per batch/lot can be increased to more than 500 as per the requirements, for example, 600, 700, 800, 900, or 1000 vials. In some embodiments, the number of vials can be 10-1000, 50-1000, 100-900, 200-800, 100-500, 100-400, 150-700, or 150-500 vials. The containers in a batch/lot can have a volume in the range of 5-100 ml, for example, such that a lot has several containers with the same volume or containers with different volumes. For example, 200 vials/containers in a batch/lot can have a volume of 10 ml each, 100 vials/containers in the same or a separate batch/lot can have a volume of 20 ml each, 100 vials/containers in the same or another batch/lot can have a volume of 30 ml each, or 300 vials/containers in the same or a different batch/lot can have a volume of 10 ml each. In some embodiments of any of the aspects and embodiments herein that include a PBMC composition, or in some compositions used in or formed by a process that includes a plurality of containers each filled with a PBMC composition comprising freeze-dried PBMCs in the form of a powder, each of the plurality of containers are purged with at least one inert gas. In some embodiments, the inert gas can be argon, or nitrogen.

Accordingly, provided herein in one aspect is a peripheral blood mononuclear cell (PBMC) composition in the form of a solid, comprising

A) a cryoprotectant, which in illustrative embodiments is or comprises trehalose, wherein in certain embodiments when the composition is rehydrated, the cryoprotectant (e.g. trehalose) is present at a concentration of from about or exactly 0.1% (w/v) to about or exactly 10.0% (w/v);

B) a lyoprotectant, which in illustrative embodiments is one, two or all of polysucrose albumin, and sorbitol, wherein in certain embodiments when the composition is rehydrated the polysucrose, albumin, and sorbitol are each, both or all in combination, present in the aqueous environment at a concentration from about or exactly 0.1% (w/v) to about or exactly 25% (w/v); and

C) a population of freeze-dried peripheral blood mononuclear cells (PBMCs), that in some embodiments is between 1×10⁵ and 1×10¹¹ freeze-dried PBMCs. In certain embodiments when the composition is rehydrated, between about or exactly 1% and 25% of the PBMCs in said population are viable. In certain embodiments either i) the composition further comprises DMSO, which in certain embodiments when the composition is rehydrated is present at a concentration between exactly or about 1% and exactly or about 5%; or ii) wherein in embodiments where sorbitol is not present, the composition further comprises PBMC cell culture media components, which in illustrative embodiments includes amino acids, vitamins, and inorganic salts. In certain embodiments, the PBMC cell culture media or the composition otherwise can further include a buffer (or the cell culture media can include an additional buffer if another buffer is present in the composition). In certain embodiments the composition when rehydrated to a target volume comprises the PBMC cell culture media components at concentrations that effectively support PBMC cell culturing and in certain most illustrative embodiments are the concentrations that are intended for such media when used for PBMC culturing.

In certain aspects, provided herein is a peripheral blood mononuclear cell (PBMC) composition in liquid form, comprising

A) a cryoprotectant, which in illustrative embodiments is or comprises trehalose, wherein in certain embodiments the cryoprotectant (e.g. trehalose) is present in the aqueous environment at a concentration of from about or exactly 0.1% (w/v) to about or exactly 10.0% (w/v);

B) a lyoprotectant, which in illustrative embodiments is one, two or all of polysucrose albumin, and sorbitol, wherein in certain embodiments the polysucrose, albumin, and sorbitol are each, both or all in combination, present at a concentration from about or exactly 0.1% (w/v) to about or exactly 25% (w/v); and

C) a population of freeze-dried peripheral blood mononuclear cells (PBMCs) suspended therein, that in some embodiments is between 1×10⁵ and 1×10¹¹ freeze-dried PBMCs.

In certain embodiments between about or exactly 1% and 25% of the PBMCs in said population are viable. In certain embodiments i) either the composition further comprises DMSO, which in certain embodiments is present at a concentration between exactly or about 1% and exactly or about 5%; or ii) wherein in embodiments where sorbitol is not present, the composition further comprises PBMC cell culture media components. In certain illustrative embodiments the PBMC cell culture media components include amino acids, vitamins, and inorganic salts, and in certain embodiments can further include a buffer (or an additional buffer if another buffer is otherwise present in the composition). In certain embodiments the composition comprises the PBMC cell culture media components at concentrations that effectively support PBMC cell culturing. In certain most illustrative embodiments the PBMC cell culture media components are at concentrations that are intended for such media when used for PBMC culturing and are effective for culturing PBMCs. In certain illustrative embodiments, the composition comprises both the sorbitol and the PBMC cell culture media components.

In one aspect, provided herein is a peripheral blood mononuclear cell (PBMC) composition in the form of a solid, comprising
A) a cryoprotectant, which in illustrative embodiments is or comprises trehalose, wherein in certain embodiments the composition is 1-50% (w/v) trehalose;
B) a lyoprotectant for example 25-75% (w/v) polysucrose and/or albumin;
C) PBMC cell culture media components comprising amino acids, vitamins, and inorganic salts which may function as a buffer, and optionally a second buffer component; and
D) a population of between 1×10⁶ and 1×10¹⁰ freeze-dried peripheral blood mononuclear cells (PBMCs), wherein at least 1% of the PBMCs in said population, when rehydrated are viable.

In illustrative embodiments, the composition when rehydrated to a target volume comprises the PBMC cell culture media components at concentrations that effectively support PBMC cell culturing.

In one aspect, provided herein is a peripheral blood mononuclear cell (PBMC) composition in the form of a solid, comprising
A) trehalose, wherein when the composition is rehydrated, the trehalose is present at a concentration of from 0.1% (w/v) to 10.0% (w/v);
B) either one or both polysucrose and albumin, wherein when the composition is rehydrated the polysucrose and albumin are independently or in combination, present at a concentration from 0.1% (w/v) to 15% (w/v);
C) DMSO in a concentration of from 1% to 5%; and
D) a population of between 1×10⁶ and 1×10¹⁰ freeze-dried peripheral blood mononuclear cells (PBMCs), wherein when the composition is rehydrated, between 1% and 25% of the PBMCs in said population are viable.

In one aspect, provided herein is a peripheral blood mononuclear cell (PBMC) composition in liquid form, comprising
A) trehalose, wherein trehalose is present at a concentration of from 0.1% (w/v) to 10.0% (w/v);
B) either one or both polysucrose and albumin, wherein when the polysucrose and albumin are independently or in combination, present at a concentration from 0.1% (w/v) to 15% (w/v);
C) DMSO in a concentration of from 1% to 5%; and
D) a population of between 1×10⁶ and 1×10¹⁰ peripheral blood mononuclear cells (PBMCs), wherein between 1% and 25% of the PBMCs in said population are viable.

In some embodiments, a PBMC composition as described herein is in the form of a solid. In some embodiments, the solid form of a PBMC composition is a powder. In some embodiments, the powder is a freeze-dried or a lyophilized powder. In some embodiments, the powder comprises less than 1% water content, or less than 1% residual moisture. In some embodiments, the powder comprises less than 0.9%, 0.8%, 0.7%, 0.6%, 0.5%, 0.4%, 0.3%, 0.2%, or 0.1% residual moisture. In some embodiments, the powder comprises 0.05-0.9%, 0.1-0.9%, 0.2-0.9%, 0.3-0.9%, 0.4-0.9%, or 0.6-0.9% residual moisture.

In some embodiments, a PBMC composition as described herein in the form of a solid comprises trehalose in a weight percentage range of 1-60%, 1-50%, 10-60%, 10-55%, 12-60%, 12-50%, 10-20%, 12-18%. In some embodiments, a PBMC composition as described herein in the form of a solid comprises polysucrose in a weight percentage of 20-80%, 25-75%, 27-80%, 25-70%, 30-70%, 50-75%, 50-70%, 60-70%, or 65-70%. In some embodiments, a PBMC composition as described herein in the form of a solid comprises albumin in a weight percentage of 0.03-75%, 0.05-70%, 0.07-65%, 0.1-1%, 0.1-0.5%, 0.5-1.0%, 10-50%, 5-60%, 10-50%, 20-40%, 20-30%, 25-30%, 26-28%, about 27%, or 27.4%. In some embodiments, a PBMC composition as described herein in the form of a solid comprises polysucrose and albumin in a combined weight percentage of 25-75%, 30-75%, 30-70%, 30-65%, 40-65%, or 45-65%, or in certain illustrative 50-70%, or 60-70%.

In some embodiments, a PBMC composition as described herein in the form of a solid comprises PBMC cell culture media components in solid form. In some embodiments, the PBMC cell culture media can be an RPMI cell culture media. In some embodiments, the PBMC cell culture media can be a customer commercial media effective for culturing PBMCs. In some embodiments, the PBMC cell culture media components comprise a buffer, amino acids, vitamins, and inorganic salts. A skilled artisan can use any well-known buffering systems for maintaining the required pH of the PBMC composition. In some embodiments, the buffer is (4-(2-hydroxyethyl)-1-piperazineethanesulphonic acid) (HEPES), sodium bicarbonate, or combinations thereof. In some embodiments, the amino acids can be a combination of two or more of L-Arginine, L-Asparagine, L-Aspartic acid, L-Cystine 2HCl, L-Glutamic Acid, L-Glutamine, L-Histidine, L-Hydroxyproline, L-Isoleucine, L-Leucine, L-Lysine hydrochloride, L-Methionine, L-Phenylalanine, L-Proline, L-Serine, L-Threonine, L-Tryptophan, L-Tyrosine disodium salt dihydrate, glutathione, and L-Valine. In some embodiments, the vitamins can be a combination of two or more of biotin, D-Calcium pantothenate, Folic Acid, Niacinamide, Para-Aminobenzoic Acid, Pyridoxine hydrochloride, Riboflavin, Thiamine hydrochloride, Vitamin B12, and i-Inositol. In some embodiments, the inorganic salts can be one or more of sodium chloride, potassium chloride, sodium citrate, disodium phosphate, monopotassium phosphate, calcium nitrate, sodium phosphate dibasic, and magnesium sulfate.

In some embodiments, a PBMC composition as described herein in the form of a solid comprises freeze-dried PBMCs in a weight percentage of 0.05-1%, 0.05-0.8%, 0.05-0.7%, 0.08-0.6%, or 0.08-0.5%. In some embodiments, a PBMC composition as described herein in the form of a solid comprises freeze-dried PBMC in a population range of between 1×10⁶ and 1×10¹¹, 1×10⁶ and 1×10¹⁰, 1×10⁶ and 1×10⁹, 1×10⁶ and 1×10⁸, or 1×10⁶ and 9×10⁷ freeze-dried PBMCs.

In some embodiments, at least 1% of PBMCs in a PBMC composition as described herein in the form of a solid are viable when the PBMC composition is rehydrated. In some embodiments, at least 0.25%, 0.4%, 0.5%, 0.6%, 0.75%, 0.8%, or 0.9% PBMCs are viable on rehydration. In some embodiments, a PBMC composition in the form of a solid that does not comprise DMSO, when rehydrated, between 1% and 9%, 1% and 8%, or 1% and 7%, of the freeze-dried PBMCs are metabolically viable. In some embodiments, a PBMC composition in the form of a solid further comprising DMSO, when rehydrated, between 10 and 25%, 12 and 25%, 15 and 25%, or 17 and 25%, freeze-dried PBMCs are metabolically viable.

In some embodiments, a PBMC composition as described herein in the form of a solid, when rehydrated to a target volume comprises the PBMC cell culture media components at concentrations that effectively support PBMC cell culturing. The target volume to which the PBMC composition in the form of a solid is to be rehydrated can be a volume in which the desired properties of the PBMC composition is preserved in the rehydrated composition. It is well-known to a skilled artisan that the PBMCs in the dried form shall preserve the characteristics which it is intended to observe once the PBMCs are rehydrated for clinical application and/or studying the characteristics such as, the presence of surface markers.

In some embodiments, a PBMC composition in the form of a solid comprises components as recited in any of the Tables 6-12. It is noteworthy that the Formula identifiers provided in the tables correspond to formulae that were tested in the Examples herein. In some embodiments, provided herein is a PBMC composition in the form of a solid having various components is recited in Table 6. In some embodiments, a PBMC composition in the form of a solid having various components is recited in Table 7. In some illustrative embodiments, a PBMC composition in the form of a solid having various components is recited in Table 8. In some embodiments, a PBMC composition in the form of a solid having various components is recited in Table 9. In some embodiments, a PBMC composition in the form of a solid having various components is recited in Table 10. In some illustrative embodiments, a PBMC composition in the form of a solid having various components is recited in Table 11. In some illustrative embodiments, a PBMC composition in the form of a solid having various components is recited in Table 12. In some embodiments, a PBMC composition in the form of a solid having various components as recited in any one of the Tables 6-12, the PBMC composition can have a variation in the range of +/−25, 20, 15, 10, 5, 4, 3, 2, or 1% in the weight percentage of any of the components as recited in the table.

In an illustrative embodiment that is supported by the data provided in the Examples for Formulation 3, provided herein is a PBMC composition in the form of a solid comprises between 10-20% trehalose, between 50-70% polysucrose, 10-20% DMSO, and dried, typically freeze-dried PBMCs, for example as 0.05-0.2% of the solid and/or between 1×10⁵ and 1×10¹¹ dried, in illustrative embodiments freeze-dried PBMCs. In another illustrative embodiment, provided herein is a PBMC composition in the form of a solid comprises between 14-18% trehalose, between 60-70% polysucrose, 12-18% DMSO, and dried, typically freeze-dried PBMCs, for example as 0.05-0.15% of the solid and/or between 1×10⁶ and 1×10¹⁰ dried, in illustrative embodiments freeze-dried PBMCs. Such illustrative embodiments supported by Formula 3 can further include a buffer at an effective amount upon rehydration of the composition, inorganic salts, for example at between 1 and 2%, additional sugars, for example dextrose, at a concentration of 0.2 to 0.6%, and a serum source, such as fetal bovine serum, at a concentration of 0.02 to 1%, or 0.04 to 0.06%. Such illustrative embodiments supported by Formula 3 can further include any of the other components provided in Table 9 at concentrations+/−20, 15, 10, 5, or 1% those provided in Formula 3. In further illustrative embodiments, between 15 and 25%, or 15 and 20% of the freeze-dried PBMCs in the compositions are viable, as demonstrated for example when rehydrated.

In an illustrative embodiment that is supported by the data provided in the Examples for Formulation 5, provided herein is a PBMC composition in the form of a solid that includes between 40-50% trehalose, between 40-50% albumin (e.g. bovine serum albumin (BSA or HSA) or human serum albumin (HSA)), and dried, typically freeze-dried PBMCs, for example as 0.1-0.3% of the solid and/or between 1×10⁵ and 1×10¹¹ dried, in illustrative embodiments freeze-dried PBMCs. In another illustrative embodiment, provided herein is a PBMC composition in the form of a solid comprises between 42-48% trehalose, between 42-48% albumin (e.g. BSA or HSA), and dried, typically freeze-dried PBMCs, for example as 0.12-0.25% of the solid and/or between 1×10⁶ and 1×10¹⁰ dried, in illustrative embodiments freeze-dried PBMCs. Such illustrative embodiments supported by Formula 5 can further include a buffer at an effective amount upon rehydration of the composition, inorganic salts, for example, at between 8 and 12%, and a serum source, such as fetal bovine serum, at a concentration of 0.02 to 0.15%, or 0.04 to 0.12%. In some embodiments, the inorganic salts include sodium chloride as 6-10%, 7-9%, or about 8% of the solid. Such illustrative embodiments supported by Formula 5 can further include any of the other components provided in Table 11 at concentrations+/−20, 15, 10, 5, or 1% those provided in Formula 5. In further illustrative embodiments, between 0.5 and 5%, or 0.75 and 2% of the freeze-dried PBMCs in the compositions are viable, as demonstrated for example when rehydrated.

In an illustrative embodiment that is supported by the data provided in the Examples for Formulation 6, provided herein is a PBMC composition in the form of a solid comprises between 10-20% trehalose, between 10-20% sorbitol, between 20 and 30% albumin, between 25 and 50% polysucrose, and dried, typically freeze-dried PBMCs, for example as 0.05-0.2% of the solid and/or between 1×10⁵ and 1×10¹¹ dried, in illustrative embodiments freeze-dried PBMCs. In another illustrative embodiment, that is supported by the data provided in the Examples for Formula 6, provided herein is a PBMC composition in the form of a solid comprises between 14-18% trehalose, between 12-18% sorbitol, between 25 and 30% albumin, between 25 and 35% polysucrose, and dried, typically freeze-dried PBMCs, for example as 0.05-0.15% of the solid and/or between 1×10⁵ and 1×10¹⁰ dried, in illustrative embodiments freeze-dried PBMCs. In illustrative embodiments, such compositions do not include any DMSO. In further embodiments of such illustrative embodiments supported by Formula 6, the freeze-dried PBMC composition further comprises PBMC cell culture media components comprising a buffer, amino acids, vitamins, and inorganic salts. Such PBMC cell culture media components can be present at concentrations that provide an effective cell culture media for culturing PBMCs when such solid freeze-dried PBMC composition is rehydrated in a target volume, which in illustrative embodiments is the volume of the composition at the start of the freeze-drying process. Such illustrative embodiments supported by Formula 6 can further include any of the other components provided in Table 12 at concentrations+/−20, 15, 10, 5, or 1% those provided in Formula 6. In some embodiments the cell culture media components are dehydrated RPMI-1640 cell culture media. In further illustrative embodiments, between 5 and 10%, or 5 and 8% of the freeze-dried PBMCs in the compositions are viable, as demonstrated for example when rehydrated.

TABLE 6

| Formulation A: | % wt |
|---|---|
| trehalose | 29.36% |
| sodium chloride | 3.40% |
| potassium chloride | 0.28% |
| HEPES | 1.76% |
| Sodium bicarbonate | 0.78% |
| dextrose | 0.81% |
| polysucrose | 62.08% |
| sodium citrate | 0.48% |
| citric acid | 0.26% |
| Bovine serum albumin | 0.26% |
| Fetal bovine serum | 0.21% |
| PBMCs | 0.32% |

TABLE 7

| Formulation 1: | % wt |
|---|---|
| trehalose | 30.67% |
| sodium chloride | 3.55% |
| potassium chloride | 0.29% |
| HEPES | 1.84% |
| Sodium bicarbonate | 0.82% |
| dextrose | 0.84% |
| polysucrose | 60.81% |
| sodium citrate | 0.51% |
| citric acid | 0.28% |
| BSA | 0.13% |
| FBS | 0.11% |
| PBMCs | 0.16% |

TABLE 8

| Formulation 2: | % wt |
|---|---|
| trehalose | 30.61% |
| sodium chloride | 3.55% |
| potassium chloride | 0.29% |
| HEPES | 1.83% |
| Sodium bicarbonate | 0.82% |
| dextrose | 0.84% |
| polysucrose | 60.68% |
| sodium citrate | 0.51% |
| citric acid | 0.28% |
| BSA | 0.13% |
| FBS | 0.11% |
| EGCG | 0.21% |
| PBMCs | 0.16% |

TABLE 9

| Formulation 3: | % wt |
|---|---|
| trehalose | 15.37% |
| sodium chloride | 1.78% |
| potassium chloride | 0.15% |
| HEPES | 0.92% |
| Sodium bicarbonate | 0.41% |
| dextrose | 0.42% |
| polysucrose | 66.03% |
| sodium citrate | 0.25% |
| citric acid | 0.14% |
| DMSO | 14.33% |
| BSA | 0.07% |
| FBS | 0.05% |
| PBMCs | 0.08% |

TABLE 10

| Formulation 4: | % wt |
|---|---|
| sodium chloride | 8.16% |
| disodium phosphate | 0.13% |
| monopotassium phosphate | 0.72% |
| trehalose | 45.32% |
| dextrose | 45.32% |
| BSA | 0.11% |
| FBS | 0.09% |
| PBMCs | 0.14% |

TABLE 11

| Formulation 5: | % wt |
|---|---|
| sodium chloride | 8.16% |
| disodium phosphate | 0.13% |
| Monopotassium phosphate | 0.72% |
| trehalose | 45.32% |
| BSA | 45.44% |
| FBS | 0.09% |
| PBMCs | 0.14% |

TABLE 12

| Formulation 6 | % wt |
|---|---|
| trehalose | 16.44850% |
| sorbitol | 16.44850% |
| bsa | 27.41416% |
| polysucrose | 32.89699% |
| Glycine | 0.00548% |
| L-Arginine | 0.10966% |
| L-Asparagine | 0.02741% |
| L-Aspartic acid | 0.01097% |
| L-Cystine 2HCl | 0.03564% |
| L-Glutamic Acid | 0.01097% |
| L-Glutamine | 0.16448% |
| L-Histidine | 0.00822% |
| L-Hydroxyproline | 0.01097% |
| L-Isoleucine | 0.02741% |
| L-Leucine | 0.02741% |
| L-Lysine hydrochloride | 0.02193% |
| L-Methionine | 0.00822% |
| L-Phenylalanine | 0.00822% |
| L-Proline | 0.01097% |
| L-Serine | 0.01645% |
| L-Threonine | 0.01097% |
| L-Tryptophan | 0.00274% |
| L-Tyrosine disodium salt dihydrate | 0.01590% |
| L-Valine | 0.01097% |
| Biotin | 0.00011% |
| Choline chloride | 0.00164% |
| D-Calcium pantothenate | 0.00014% |
| Folic Acid | 0.00055% |
| Niacinamide | 0.00055% |
| Para-Aminobenzoic Acid | 0.00055% |
| Pyridoxine hydrochloride | 0.00055% |
| Riboflavin | 0.00011% |
| Thiamine hydrochloride | 0.00055% |
| Vitamin B12 | 0.000003% |
| i-Inositol | 0.01919% |
| Calcium nitrate (Ca(NO₃)₂ 4H₂O) | 0.05483% |
| Magnesium Sulfate (MgSO₄) (anhyd.) | 0.02678% |
| Potassium Chloride (KCl) | 0.21931% |
| Sodium Bicarbonate (NaHCO₃) | 1.09657% |
| Sodium Chloride (NaCl) | 3.28970% |
| Sodium Phosphate dibasic (Na₂HPO₄) | 0.43863% |
| D-Glucose (Dextrose) | 1.09657% |
| Glutathione (reduced) | 0.00055% |
| FBS | 0.05612% |
| PBMCs | 0.08537% |

Further exemplary aspects and embodiments of PBMC compositions are provided throughout this specification. As an example, such PBMC compositions are provided in the Exemplary Embodiments section.

Processes/Methods for Preparing Freeze-Dried Platelets

In some embodiments and aspects provided herein are processes/methods for preparing freeze-dried (e.g., lyophilized) PBMCs and for preparing rehydrated PBMCs that had been freeze-dried. The processes/methods typically include the following steps:

A) incubating PBMCs in a liquid, typically an aqueous solution that includes a cryoprotectant, which in illustrative embodiments is trehalose and/or DMSO, and a lyoprotectant, that in illustrative embodiments is polysucrose, DMSO, and/or albumin; and
B) lyophilizing the liquid to form freeze-dried PBMCs.

The aqueous solution is typically an aqueous mixture in an aqueous environment, which typically is a buffered aqueous solution such as a buffered aqueous mixture in a buffered aqueous environment. Details regarding components of the aqueous mixture and time and temperatures for the incubation are provided herein. Furthermore, various aspects and embodiments of such processes/methods are provided herein.

In some embodiments, provided herein is a method of preparing freeze-dried PBMCs comprising incubating PBMCs in a buffered aqueous environment that includes at least a buffer, a salt, and a sugar, and optionally a bulking agent to form a mixture, and lyophilizing the mixture to form freeze-dried PBMCs.

In some embodiments, provided herein is a method of preparing freeze-dried PBMCs comprising incubating PBMCs with trehalose in an aqueous solution, such as a buffered aqueous environment that includes at least a buffer, a salt, optionally a sugar, and optionally a bulking agent to form a mixture, and lyophilizing the mixture to form freeze-dried PBMCs.

In some embodiments, provided herein is a process for preparing freeze-dried (e.g., lyophilized) PBMCs, comprising: incubating PBMCs in a buffered aqueous solution that comprises at least a buffer, a salt, and a sugar, to form a first mixture. The buffered aqueous solution can include a cryoprotectant, such as trehalose.

In some embodiments, provided herein is a process for preparing freeze-dried (e.g., lyophilized) PBMCs, comprising incubating PBMCs with trehalose in an aqueous solution, such as a buffered aqueous environment that includes at least a buffer, a salt, optionally a sugar, and optionally a bulking agent to form a first mixture.

In some embodiments a bulking/stabilizing agent, such as polysucrose, can be present during incubation. In some embodiments the process can include contacting the incubated PBMCs with a bulking/stabilizing agent, such as polysucrose, to form a second mixture. In some embodiments, the process includes lyophilizing a PBMC-containing mixture to form freeze-dried PBMCs. In some embodiments, polysucrose is added to the buffered aqueous environment in which the PBMCs are then incubated. In some embodiments, the PBMCs are incubated in the buffered aqueous environment to form a mixture, after which polysucrose is added to the mixture.

In some more particular embodiments, provided herein is a process for preparing freeze-dried (e.g., lyophilized) PBMCs, comprising: incubating the PBMCs in a buffered aqueous solution that includes at least a buffer, a salt, and trehalose to form incubated PBMCs to form a first mixture; and lyophilizing the first mixture to form freeze-dried PBMCs. In some more particular embodiments, the first mixture comprises polysucrose.

In some more particular embodiments, provided herein is a process for preparing freeze-dried (e.g., lyophilized) PBMCs, comprising: incubating the PBMCs in a buffered aqueous solution that includes at least a buffer, a salt, trehalose, and dextrose to form incubated PBMCs to form a first mixture; and lyophilizing the first mixture to form freeze-dried PBMCs. In some more particular embodiments, the first mixture comprises polysucrose.

In some embodiments, the process includes incubating the PBMCs for any of the incubating times and temperatures provided herein, with a first mixture comprising trehalose, sorbitol, and albumin (e.g. bovine serum albumin (BSA)) in an aqueous environment to form incubated PBMCs, contacting the incubated PBMCs with polysucrose to form a second mixture, and lyophilizing the second mixture to form freeze-dried PBMCs. The first mixture can contain a buffer. In some embodiments the buffer is present in albumin (e.g. BSA). In some embodiments, the buffer present in BSA is PBS. In some embodiments, the buffer is sodium bicarbonate.

In embodiments of any of the methods/processes and compositions, provided herein, the aqueous mixture and/or the rehydrated platelet composition comprises a PBMC cell culture media/medium, or components thereof. Thus, in some embodiments of preparing freeze-dried PBMCs, the incubating buffer comprises a cell culture medium. In some embodiments, the cell culture medium is a cell culture medium comprising one or more, or typically all of the following components: vitamins (e.g., biotin, vitamin B12, (para-aminobenzoic acid (PABA), inositol, choline), amino acids, salts, and a buffer(s) (e.g. sodium bicarbonate), and combinations thereof. In some embodiments, the cell culture medium is Roswell Park Memorial Institute (RPMI) cell culture medium. In some embodiments, the RPMI cell culture medium is RPMI-1640 cell culture media. It will be understood that when PBMCs are replaced with other nucleated cell types in the methods/processes and compositions herein, some embodiments include cell culture media that support culturing of such other nucleated cell types.

In some embodiments, when dried compositions provided herein, are rehydrated to a target volume, the resulting liquid composition comprises the PBMC cell culture media components at concentrations that effectively support PBMC cell culturing, and in illustrative embodiments are at their target concentrations for that particular media. In some embodiments of freeze-dried PBMCs prepared by the any of the processes described herein, the process comprises admixing the cell culture media (e.g. RPMI-1640) into a mixture that can include other components as indicated herein. In some embodiments, such admixing is performed before PBMCs are freeze-dried. In other embodiments, such admixing is performed to rehydrate freeze-dried PBMCs.

In further embodiments, the cell culture media components can be selected from those found in Roswell Park Memorial Institute medium (RPMI) 1640 medium, Iscove's modified Dulbecco's medium (IMDM), Dulbecco's modified Eagle medium (DMEM), McCoy's 5 A medium, minimum essential medium alpha medium (alpha-MEM), basal medium Eagle (BME), Fischer's medium, medium 199, and F-12K nutrient mixture medium (Kaighn's modification, F-12K). In some embodiments, the cell culture media can be any cell culture media that supports growth of PBMCs or any cell type therein (e.g. T cell culture media, NK cell culture media, B cell culture media, or monocyte cell culture media). In some embodiments, the cell culture media is a custom media developed for PBMCs and/or any cell type therein. In certain illustrative embodiments, the cell culture media is RPMI-1640 cell culture media. In some embodiments, the first mixture contains RPMI-1640, which contains sodium bicarbonate. An example of the RPMI-1640 formulation/components is provided at www.thermofisher.com/us/en/home/technical-resources/media-formulation. 115 html, incorporated herein in its entirety. The RPMI-1640 formulation is provided below in the table of RPMI components and is expressly incorporated herein.

Table of RPMI components

| | | | |
|---|---|---|---|
| | Molecular | Concentration | |
| Components | Weight | (mg/L) | mM |

Amino Acids

| | | | |
|---|---|---|---|
| Glycine | 75 | 10 | 0.133333 |
| L-Arginine | 174 | 200 | 1.149425 |
| L-Asparagine | 132 | 50 | 0.378788 |
| L-Aspartic acid | 133 | 20 | 0.150376 |
| L-Cystine 2HCl | 313 | 65 | 0.207668 |
| L-Glutamic Acid | 147 | 20 | 0.136054 |
| L-Glutamine | 146 | 300 | 2.054795 |
| L-Histidine | 155 | 15 | 0.096774 |
| L-Hydroxyproline | 131 | 20 | 0.152672 |
| L-Isoleucine | 131 | 50 | 0.381679 |
| L-Leucine | 131 | 50 | 0.381679 |
| L-Lysine hydrochloride | 183 | 40 | 0.218579 |
| L-Methionine | 149 | 15 | 0.100671 |
| L-Phenylalanine | 165 | 15 | 0.090909 |
| L-Proline | 115 | 20 | 0.173913 |
| L-Serine | 105 | 30 | 0.285714 |
| L-Threonine | 119 | 20 | 0.168067 |
| L-Tryptophan | 204 | 5 | 0.02451 |
| L-Tyrosine disodium salt | 261 | 29 | 0.111111 |
| dihydrate | | | |
| L-Valine | 117 | 20 | 0.17094 |

Vitamins

| | | | |
|---|---|---|---|
| Biotin | 244 | 0.2 | 8.20E−04 |
| Choline chloride | 140 | 3 | 0.021429 |
| D-Calcium pantothenate | 477 | 0.25 | 5.24E−04 |
| Folic Acid | 441 | 1 | 0.002268 |
| Niacinamide | 122 | 1 | 0.008197 |
| Para-Aminobenzoic Acid | 137 | 1 | 0.007299 |
| Pyridoxine hydrochloride | 206 | 1 | 0.004854 |
| Riboflavin | 376 | 0.2 | 5.32E−04 |
| Thiamine hydrochloride | 337 | 1 | 0.002967 |
| Vitamin B12 | 1355 | 0.005 | 3.69E−06 |
| i-Inositol | 180 | 35 | 0.194444 |

Inorganic Salts

| | | | |
|---|---|---|---|
| Calcium nitrate | 236 | 100 | 0.423729 |
| (Ca(NO3)2 4H2O) | | | |
| Magnesium Sulfate | 120 | 48.84 | 0.407 |
| (MgSO4) (anhyd.) | | | |
| Potassium Chloride | 75 | 400 | 5.333334 |
| (KCl) | | | |
| Sodium Bicarbonate | 84 | 2000 | 23.80953 |
| (NaHCO3) | | | |
| Sodium Chloride (NaCl) | 58 | 6000 | 103.4483 |
| Sodium Phosphate | 142 | 800 | 5.633803 |
| dibasic (Na2HPO4) | | | |

Other Components

| | | | |
|---|---|---|---|
| D-Glucose (Dextrose) | 180 | 2000 | 11.11111 |
| Glutathione (reduced) | 307 | 1 | 0.003257 |

In some embodiments, provided herein is a process for preparing freeze-dried (e.g., lyophilized) PBMCs, comprising incubating PBMCs in an incubating buffer to form a first mixture; contacting the first mixture with polysucrose to form a second mixture, and lyophilizing the second mixture to form freeze-dried (e.g., lyophilized) PBMCs. In some embodiments, the incubating buffer comprises trehalose. In some embodiments, the incubating buffer comprises sorbitol. In some embodiments, the incubating buffer comprises BSA.

In some embodiments, a PBMC composition that includes freeze-dried PBMCs in the form of a liquid, or a PBMC composition that includes freeze-dried PBMCs in the form of a solid, when rehydrated, preserve the cell surface markers of PBMCs at a level of at least 10%, 15, 20%, 25%, 30%, 40%, 50%, 60%, 70%, or 75% of fresh PBMCs. In some embodiments, the PBMC composition as described herein preserve the cell surface markers of PBMCs at a level in the range of 10-99%, 15-90%, 20-85%, or 25-80% of fresh PBMCs. In some embodiments, the freeze-dried PBMCs have an increased level of surface marker preservation for some of the surface markers as compared to PBMCs that are not incubated with the incubating buffer or that are not processed according methods for preparing freeze-dried PBMCs provided herein. In some embodiments, the surface markers that are preserved are CD19, CD45, CD3, CD14, CD4, CD8, CD56. In some embodiments, in a PBMC composition comprising freeze-dried PBMCs as disclosed herein, the cell surface markers on the freeze-dried PBMCs are preserved as compared to fresh PBMCs. In some embodiments, in a PBMC composition comprising freeze-dried PBMCs as described herein, at least 5% of the freeze-dried PBMCs are positive for CD 19, and in fresh PBMCs used for comparison, at least 13% of fresh PBMCs are positive for CD 19. In some embodiments, 3-20%, 5-20%, 5-18%, or 5.25-15% of the freeze-dried PBMCs are positive for CD 19. In some embodiments, at least 45% of the freeze-dried PBMCs are positive for CD 45, and in fresh PBMCs used for comparison, at least 68% of fresh PBMCs are positive for CD 45. In some embodiments, 40-70%, 42-68%, 45-67%, or 46-66% of the freeze-dried PBMCs are positive for CD 45. In some embodiments, at least 45% of the freeze-dried PBMCs are positive for CD 3, and in fresh PBMCs used for comparison, at least 60% of fresh PBMCs are positive for CD 3. In some embodiments, 40-70%, 42-68%, or 44-66% of the freeze-dried PBMCs are positive for CD 3. In some embodiments, at least 8% of the freeze-dried PBMCs are positive for CD 14, and in fresh PBMCs used for comparison, at least 18% of fresh PBMCs are positive for CD 14. In some embodiments, 7-25%, 8-23%, or 10-18% of the freeze-dried PBMCs are positive for CD 14. In some embodiments, at least 45% of the freeze-dried PBMCs are positive for CD 4, and in fresh PBMCs used for comparison, at least 45% of fresh PBMCs are positive for CD 4. In some embodiments, 40-70%, 42-67%, or 44-67%, of the freeze-dried PBMCs are positive for CD 4. In some embodiments, at least 10% of the freeze-dried PBMCs are positive for CD 8, and in fresh PBMCs used for comparison, at least 25% of fresh PBMCs are positive for CD 8. In some embodiments, 10-35%, 11-30%, or 12-27% of the freeze-dried PBMCs are positive for CD 8. In some embodiments, at least 4% of the freeze-dried PBMCs are positive for CD 56, and in fresh PBMCs used for comparison, at least 20% of fresh PBMCs are positive for CD 56. In some embodiments, 3-25%, 5-23%, or 5-20% of the freeze-dried PBMCs are positive for CD 56. In some embodiments, in a PBMC composition comprising freeze-dried PBMCs as described herein, the freeze-dried PBMCs display more than 20%, 30%, 40%, 50%, 60%, 65%, 70%, or 75% of surface marker positivity relative to fresh PBMCs, wherein the surface marker is selected from the group consisting of CD45, CD3, CD14, CD4, CD8, CD56. In some embodiments, the freeze-dried PBMCs display 20-95%, 25-90%, 30-85%, or 25-80% of surface marker positivity relative to fresh PBMCs, wherein the surface marker is selected from the group consisting of CD45, CD3, CD14, CD4, CD8, CD56. The surface marker positivity of the freeze-dried PBMCs relative to fresh PBMCs as described herein is calculated by dividing a surface marker positivity value of freeze-dried PBMCs with the surface marker positivity value of fresh PBMCs and multiplying the obtained result with “100”. In some embodiments, in a PBMC composition comprising freeze-dried PBMCs as described herein, the freeze-dried PBMCs, display more than 30%, 35%, 40%, 45%, or 50% total mean fluorescent intensity (MFI) of a surface marker relative to fresh PBMCs, wherein the surface marker is selected from the group consisting of CD45, CD3, CD14, CD4, CD8, CD56. In some embodiments, the freeze-dried PBMCs display 30-75%, 35-70%, or 35-60% total MFI of a surface marker relative to fresh PBMCs, wherein the surface marker is selected from the group consisting of CD45, CD3, CD14, CD4, CD8, CD56. The relative MFI percentage of the freeze-dried PBMCs is calculated by dividing an MFI value of a surface marker of freeze-dried PBMCs with the MFI value of the surface marker of fresh PBMCs and multiplying the obtained result with “100”. In some embodiments, in a PBMC composition comprising freeze-dried PBMCs as described herein, the freeze-dried PBMCs show high positivity for CD 4 than compared to fresh PBMCs. In some embodiments, the freeze-dried PBMCs show at least 0.1%, 1%, 2%, 3%, 4%, 5%, 7%, 8%, 10%, 12%, or 15% high CD 4 positivity than compared to fresh PBMCs. In some embodiments, the freeze-dried PBMCs show 0.1-20%, 1-17%, 2-17%, or 3-15% high CD 4 positivity compared to fresh PBMCs. It would be understood that the positivity for cell surface markers was measured using flow cytometry. In some embodiments, in a PBMC composition comprising freeze-dried PBMCs as described herein, the freeze-dried PBMCs show high number of CD 4 present on them as compared to fresh PBMCs. In some embodiments, the freeze-dried PBMCs show at least 2%, 3%, 4%, 5%, 7%, 8%, 10%, 12%, or 15% more number of CD 4 markers present on them as compared to fresh PBMCs. In some embodiments, the freeze-dried PBMCs show 2-20%, 3-17%, or 5-15% more number of CD 4 markers present on them as compared to fresh PBMCs. It is to be understood that the number of cell surface markers, for example, CD 4 was measured using the antigen specific antibodies. In some embodiments, the freeze-dried PBMCs retain functions and are capable of responding to biochemical signaling similar to fresh PBMCs.

In some embodiments of a process for preparing a PBMC composition as disclosed herein or a PBMC composition as disclosed herein, the composition comprises lymphocytes. In some embodiments, the lymphocytes comprise any one of T cells, B cells, NK cells, or combinations thereof. In some embodiments of a process for preparing a PBMC composition as disclosed herein or a PBMC composition as described herein, the composition comprises monocytes. In some embodiments, the process as disclosed herein can comprise preparing a composition comprising monocytes, lymphocytes, or combinations thereof.

In some embodiments, a PBMC composition comprising freeze-dried PBMCs as described herein can comprise a population of T cells, wherein at least 45% of the freeze-dried PBMCs are positive for CD 4. In some embodiments, the freeze-dried PBMCs are 40-75%, 45-70%, or 40-65% positive for CD 4. In some embodiments, a PBMC composition comprising freeze-dried PBMCs as disclosed herein can include a population of T cells, wherein at least 10% of the freeze-dried PBMCs are positive for CD 8. In some embodiments, the freeze-dried PBMCs are 10-35%, 12-30%, or 12-27% positive for CD 8. It would be understood that CD 4 and CD8 are markers for T cells, and in some embodiments, the percent positivity can reflect the percentage of T cells present in a PBMC composition.

In illustrative embodiments, compositions and methods of preparing, using and administering the same herein include all of the cell types found in PBMCs. However, in some aspects, rather than a PBMC composition comprising PBMCs or freeze-dried PBMCs, or a method of preparing, using, or administering PBMCs or freeze-dried PBMCs, compositions and methods of preparing, using, or administering the same herein, include one, or some but not all of the cell types in PBMCs. Thus, in some aspects provided herein, for example are T cell compositions comprising freeze-dried T cells, NK cell compositions comprising freeze-dried NK cells, B cell compositions comprising freeze-dried B cells, monocyte compositions comprising freeze-dried monocytes, and NK/T compositions comprising freeze-dried T cells and NK cells. Furthermore, aspects herein include methods of making, using, or administering the same. In some embodiments of any of the compositions or methods herein, the PBMCs are genetically-modified PBMCs, the T cells are genetically-modified T cells, the NK cells are genetically-modified NK cells, the B cells are genetically-modified B cells, and/or the monocytes are genetically-modified monocytes. For aspects and embodiments that include one, or some but not all of the cell types of PBMCs, a skilled artisan will understand which of those surface markers are expressed on those cell types, and based on the surface marker data herein, in illustrative embodiments of freeze-dried cells produced using methods herein, how much of those relevant cell markers for that cell type are retained on freeze-dried versions of that cell type.

In some embodiments, a PBMC composition comprising freeze-dried PBMCs as described herein can comprise a population of B cells, wherein at least 4% of the freeze-dried PBMCs are positive for CD 19. In some embodiments, the freeze-dried PBMCs are 4-15%, 5-15%, or 5-14% positive for CD 19. It would be understood that CD 19 is a marker for B cells, and in some embodiments, the percent positivity can reflect the percentage of B cells present in a PBMC composition. In some embodiments of a PBMC composition comprising freeze-dried PBMCs, such a PBMC composition can be a B cell composition comprising freeze-dried B cells. In some embodiments, the B cells can be genetically modified B cells.

In some embodiments, a PBMC composition comprising freeze-dried PBMCs as described herein can comprise a population of NK cells, wherein at least 45% of the freeze-dried PBMCs are positive for CD 3. In some embodiments, the freeze-dried PBMCs are 50-75%, 45-75%, or 45-70% positive for CD 3. In some embodiments, a PBMC composition comprising freeze-dried PBMCs as described herein can comprise a population of NK cells, wherein at least 5% of the freeze-dried PBMCs are positive for CD 56. In some embodiments, the freeze-dried PBMCs are 4-30%, 5-25%, or 5-22% positive for CD 56. It would be understood that CD 3 and/or CD 56 is/are marker(s) for NK cells, and in some embodiments, the percent positivity can reflect the percentage of NK cells present in a PBMC composition. In some embodiments of a PBMC composition comprising freeze-dried PBMCs, such a PBMC composition can be a NK cell composition comprising freeze-dried NK cells. In some embodiments, the NK cells can be genetically modified NK cells.

In some embodiments of a process for preparing a PBMC composition comprising freeze-dried PBMCs as described herein, such a process can be used for preparing a composition comprising freeze-dried T cells. In some embodiments, the process can be used for preparing a composition comprising freeze-dried B cells. In some embodiments, the process can be used for preparing a composition comprising freeze-dried NK cells. In some embodiments, the process can be used for preparing a composition comprising freeze-dried monocytes. In some embodiments, the process can be used for preparing a composition comprising any one or more of freeze-dried B cells, freeze-dried T cells, freeze-dried NK cells, and freeze-dried monocytes. It can be contemplated that in some embodiments, a process for preparing a PBMC composition comprising freeze-dried PBMCs can also be used to prepare a composition comprising freeze-dried cells of any one type of PBMCs selected from the group consisting of T cells, B cells, NK cells, and monocytes. In some embodiments, the T cells, B cells, NK cells, and monocytes can be genetically modified, such as genetically modified types of T cells, genetically modified types of B cells, genetically modified types of NK cells, and genetically modified types of monocytes. It further can be contemplated that the process for preparing a composition comprising freeze-dried cells of any type of PBMC as described herein preserves the respective surface markers of each cell types, for example, in one illustrative embodiment, the process for preparing freeze-dried T cells as described herein preserves the CD 4 ad CD 8 markers.

In some embodiments, the process includes incubating the PBMCs in a mixture comprising a buffer (such as PBS or a buffer in the cell culture media, for example RPMI-1640), trehalose, and bovine serum albumin (BSA) in an aqueous environment to form incubating PBMCs. In some embodiments, a bulking/stabilizing agent, such as polysucrose and/or sorbitol, may be added to the incubated PBMCs formed in the above step to form a mixture. The mixture is then lyophilized to form freeze-dried PBMCs. In some embodiments, a bulking/stabilizing agent, such as polysucrose and/or sorbitol, can be present during the incubating of the PBMCs, for example in the aqueous environment. The incubated PBMCs are then lyophilized to form freeze-dried PBMCs.

Thus, provided herein, are populations of freeze-dried peripheral mononuclear blood cells (PBMCs), wherein some PBMCs of said population, when rehydrated are viable. For example, in some embodiments the population has a viability level of at least 1%. In some embodiments, the population of freeze-dried PBMCs has a viability level of at least 3%. In some embodiments, the population has a viability level of at least 5%. Further embodiments that provide percent viability of such PBMC populations can be found in this disclosure.

In some embodiments, any type of nucleated cell from any source can be freeze-dried using the methods herein, and thus in some aspects provided herein are freeze-dried compositions and rehydrated compositions comprising such freeze-dried nucleated cells that include other components as discussed herein for illustrative embodiments, with respect to PBMCs. Accordingly, in some embodiments, nucleated cells in the compositions and methods herein are PBMCs. In some embodiments, the nucleated cells (e.g. PBMCs) and/or freeze-dried nucleated cells (e.g. PBMCs) are mammalian cells. In some embodiments, the freeze-dried nucleated cells (e.g. PBMCs) are from an animal source, for example, canine, bovine, equine, or feline. In illustrative embodiments, the freeze-dried nucleated cells (e.g. PBMCs) are human cells.

In some embodiments, the PBMCs and/or freeze-dried PBMCs are lymphocytes or monocytes. In some embodiments, the lymphocytes are B-cells or T-cells. In some embodiments, the lymphocytes are recombinant lymphocytes. For example, the lymphocytes can be CAR-T cells, T cell comprising a recombinant T cell receptor, or genetically engineered NK cells. Thus, such T cells or NK cells can contain nucleic acids that encode a chimeric antigen receptor. Furthermore, in some embodiments, the cells that are freeze-dried in the process or that are present in dried form or rehydrated form, are granulocytes. In some embodiments, the cells that are freeze-dried in the process or that are present in dried form or rehydrated form, are stem cells.

Thus, provided herein are processes of preparing freeze-dried PBMCs, the process including the following steps:

incubating PBMCs with a first mixture comprising trehalose, sorbitol, and BSA in an aqueous environment to form incubated PBMCs; contacting the incubated PBMCs with polysucrose to form a second mixture; and lyophilizing the second mixture to form freeze-dried PBMCs.

In some embodiments, provided herein is a process for preparing freeze-dried (e.g., lyophilized) PBMCs, comprising: incubating/contacting PBMCs with a mixture comprising polysucrose, trehalose, sorbitol, and BSA in an aqueous environment to form a first mixture; and lyophilizing the first mixture to form freeze-dried PBMCs.

Thus, provided herein, is a method of preparing freeze-dried PBMCs, the method comprising: incubating PBMCs with a mixture comprising 6% (w/v) polysucrose, 3% (w/v) trehalose, 3% (v/v) sorbitol, and 5% (w/v) BSA in an aqueous environment to form incubated PBMCs; and lyophilizing the mixture to form freeze-dried PBMCs.

Thus, provided herein are freeze-dried PBMCs prepared by a process comprising the steps of: incubating PBMCs with a mixture comprising 6% (w/v) polysucrose, 3% (w/v) trehalose, 3% (v/v) sorbitol, and 5% (w/v) BSA in an aqueous environment to form incubated PBMCs; and lyophilizing the mixture, to form freeze-dried PBMCs.

Thus, provided herein is a method of preparing freeze-dried PBMCs, the method comprising: incubating PBMCs with a mixture comprising polysucrose, trehalose, and DMSO in an aqueous environment to form incubated PBMCs; and lyophilizing the mixture to form freeze-dried PBMCs.

Thus, provided herein is a method of preparing freeze-dried PBMCs, the method comprising: incubating PBMCs with a mixture comprising 13% (w/v) polysucrose, 3% (w/v) trehalose, and 2.5% (v/v) DMSO in an aqueous environment to form incubated PBMCs; and lyophilizing the mixture to form freeze-dried PBMCs.

Thus, provided herein, is a method of preparing freeze-dried PBMCs, the method comprising: incubating PBMCs with a mixture comprising trehalose and BSA in an aqueous environment to form incubated PBMCs; and lyophilizing the mixture to form freeze-dried PBMCs.

Thus, provided herein, is a method of preparing freeze-dried PBMCs, the method comprising: incubating PBMCs with a mixture comprising 5% (w/v) trehalose and 5% (w/v) BSA in an aqueous environment to form incubated PBMCs; and lyophilizing the mixture to form freeze-dried PBMCs.

In some embodiments of the processes for preparing freeze-dried PBMCs, the trehalose is present in the aqueous environment at a concentration of from about 0.1% (w/v) to about 10.0% (w/v). In some embodiments of the processes for preparing freeze-dried PBMCs, the trehalose is present in the aqueous environment at a concentration of from about 1.0% (w/v) to about 5.0% (w/v). In some embodiments of the processes for preparing freeze-dried PBMCs, the sorbitol is present in the aqueous environment at a concentration from about 0.1% (w/v) to about 5.0% (w/v). In some embodiments of the processes for preparing freeze-dried PBMCs, the BSA is present in the aqueous environment at a concentration from about 0.1% (w/v) to about 10.0% (w/v). In some embodiments of the processes for preparing freeze-dried PBMCs, the BSA is present in the aqueous environment at a concentration from about 3.0% (w/v) to about 7.0% (w/v). In some embodiments of the processes for preparing freeze-dried PBMCs, the polysucrose is present in the aqueous solution at a concentration from about 0.1% (w/v) to about 25% (w/v), such as from about 0.1% (w/v) to about 17% (w/v), such as from about 0.1% (w/v) to about 13% (w/v), such as from about 0.1% (w/v) to about 10% (w/v). In some embodiments of the processes for of preparing freeze-dried PBMCs, the polysucrose is present in the aqueous solution at a concentration from about 3.0% (w/v) to about 7.0% (w/v).

Thus, provided herein are rehydrated freeze-dried PBMC produced by any of the methods described herein, where the method comprises rehydrating the lyophilized PBMCs. In some embodiments, cell culture medium, or

Thus, provided herein are freeze-dried PBMCs prepared by a process comprising the steps of:

incubating PBMCs with a first mixture comprising trehalose, sorbitol, and BSA in an aqueous environment to form incubated PBMCs;
contacting the incubated PBMCs with polysucrose to form a second mixture; and lyophilizing the second mixture, to form freeze-dried PBMCs.

Thus, provided, herein are freeze-dried PBMCs are prepared by a process comprising the steps of incubating PBMCs in an aqueous environment that includes at least a buffer, a salt, and a sugar, and optionally a bulking agent to form a mixture and lyophilizing the mixture to form freeze-dried PBMCs.

Thus, provided herein are freeze-dried PBMCs prepared by a process comprising the steps of:

incubating/contacting PBMCs with a mixture comprising polysucrose, trehalose, sorbitol, and BSA in an aqueous environment to form a first mixture; and lyophilizing the first mixture to form freeze-dried PBMCs.

Thus, provided herein are freeze-dried PBMCs prepared by a process comprising the steps of: incubating PBMCs with a mixture comprising polysucrose, trehalose, and DMSO in an aqueous environment to form incubated PBMCs; and lyophilizing the mixture, to form freeze-dried PBMCs.

Thus, provided herein are freeze-dried PBMCs prepared by a process comprising the steps of: incubating PBMCs with a mixture comprising trehalose and BSA in an aqueous environment to form incubated PBMCs; and lyophilizing the mixture, to form freeze-dried PBMCs.

In some embodiments of freeze-dried PBMCs prepared by the any of the processes described herein, the trehalose is present in the aqueous environment at a concentration of from about 0.1% (w/v) to about 10.0% (w/v), the sorbitol is present in the aqueous environment at a concentration from about 0.1% (w/v) to about 5.0% (w/v);

the BSA is present in the aqueous environment at a concentration from about 0.1% (w/v) to about 10.0% (w/v), and the polysucrose is present in the aqueous solution at a concentration from about 0.1% (w/v) to about 25% (w/v), such as from about 0.1% (w/v) to about 17% (w/v), such as from about 0.1% (w/v) to about 13% (w/v), such as from about 0.1% (w/v) to about 10% (w/v).

In some embodiments of freeze-dried PBMCs prepared by the any of the processes described herein, the trehalose is present in the aqueous environment at a concentration of from about 1.0% (w/v) to about 5.0% (w/v), the sorbitol is present in the aqueous environment at a concentration of from about 2.5% to about 3.5%, the BSA is present in the aqueous environment at a concentration from about 3.0% (w/v) to about 7.0% (w/v), and the polysucrose is present in the aqueous solution at a concentration from about 3.0% (w/v) to about 7.0% (w/v).

In some embodiments of freeze-dried PBMCs prepared by the any of the processes described herein, the population has a viability level of at least 1%. In some embodiments of freeze-dried PBMCs prepared by the any of the processes described herein, the population has a viability level of at least 3%. In some embodiments of freeze-dried PBMCs prepared by the any of the processes described herein, the population has a viability level of at least 5%. In some embodiments of freeze-dried PBMCs prepared by the any of the processes described herein, between 1% and 25%, for example, between 1 and 20%, between 5 and 20%, between 1 and 10%, or between 1 and 8% of the freeze-dried PBMCs are metabolically viable.

In some embodiments of a PBMC composition comprising freeze-dried PBMCs prepared by the any of the processes described herein that further comprises DMSO, between 10 and 25%, 12 and 25%, 15 and 25%, or 17 and 25%, freeze-dried PBMCs are metabolically viable.

In some embodiments of a PBMC composition comprising freeze-dried PBMCs prepared by the any of the processes described herein that does not comprise DMSO, between 1% and 10% of the freeze-dried PBMCs are metabolically viable. In some embodiments, a PBMC composition does not comprise DMSO, and such a composition in the liquid form or when the composition is in the form of a solid is rehydrated, between 1% and 9%, 1% and 8%, or 1% and 7%, of the freeze-dried PBMCs are metabolically viable.

The process of preparing lyophilized PBMCs optionally includes obtaining or preparing the PBMCs for incubating and lyophilization. Obtaining or preparing the PBMCs can be any action that results in providing purified or isolated PBMCs for subsequent use in the process. For example, PBMCs can be obtained from a vendor (e.g., e.g., StemExpress, ATCC, Stemcell Technologies, or Hemacare). Additionally, PBMCs may be isolated by any of several standard techniques, including, but not limited to: centrifugation, tissue culture, affinity column binding, fluorescence-activated cell sorting (FACS), filtration, or other techniques standard in the art. Within the context of blood cells, many suitable protocols can be used for separating white blood cells from red blood cells, platelets, and plasma, and any such protocols can be used. Preferably, a protocol that involves centrifugation of whole blood to separate the various components from each other is used. For example, the commercially available BD brand CPT blood draw tube can be used for centrifugation-driven separation of white blood cells from other blood components. In general, for centrifugation-driven separation of white blood cells, conditions of centrifugation at room temperature for 25 minutes at 1,700×g, or equivalent conditions, are suitable. PBMCs can be extracted from whole blood using polysucrose and gradient centrifugation, which separates blood into a top layer of plasma, followed by a layer of PBMCs and a bottom fraction of polymorphonuclear cells and erythrocytes. Cells separated from other cells or biological material can be washed one or more times to enhance purity.

In some embodiments the process includes incubating the PBMCs with a mixture, typically an aqueous mixture, that includes a cryoprotectant, for example to form incubated, heat-treated, and/or cryoprotected PBMCs suspended in the mixture. Incubating of the PBMCs results from contacting the PBMCs with a cryoprotectant for an amount of time and under appropriate conditions whereby the cryoprotectant is taken up by the PBMCs. Contacting thus can be exposing the PBMCs to the cryoprotectant by combining, mixing, etc. the two in an aqueous environment. Incubating the PBMCs with a cryoprotectant is believed to protect the PBMCs from lysis and to promote retention of viability during lyophilization and rehydration. The cryoprotectant can be any of the known substances suitable for protection during lyophilization of cells, such as platelets. Exemplary embodiments include the use of a sugar, such as trehalose. Exemplary embodiments include the use of a cryoprotective solvent, such as DMSO. While not being bound by any particular mode of operation, entry of the cryoprotectant into the cells is believed to occur through a process of thermal endocytosis. In some embodiments, for incubating the PBMCs with trehalose, the PBMCs are exposed to trehalose from 30 minutes to four hours at a temperature of between 20° C. and 40° C., such as from one to four hours at a temperature of between 25° C. and 40° C. In exemplary embodiments, the PBMCs are incubated in the presence of trehalose for 30 minutes to 2 hours at 37° C. In exemplary embodiments, the PBMCs are incubated in the presence of trehalose for 30 minutes at 37° C. Optionally, the combination of PBMCs and cryoprotectant can be gently agitated, such as by inversion of the incubation chamber, periodically, such as every 10-30 minutes, such as every 10 minutes, or such as every 30 minutes. In illustrative embodiments, PBMCs are suspended in a lyoprotectant before being freeze-dried. Such lyoprotectant can include, as non-limiting examples, one or more proteins, such as albumin, polysucrose, or a derivative thereof, and/or a polyol such as sorbitol.

In some embodiments the incubating composition is an aqueous solution of at least the PBMCs and the cryoprotectant (e.g., sugar). Thus, in some embodiments, PBMCs are suspended in an aqueous solution that includes a cryoprotectant (e.g., sugar). PBMCs can be processed such that they are suspended in the aqueous solution using methods such as centrifugation and tangential flow filtration. In some embodiments, trehalose is used as the cryoprotectant, and it is present in an amount of from about 0.1% (w/v) to about 15% (w/v), from about 0.5% (w/v) to about 14% (w/v), from about 1% (w/v) to about 13% (w/v), from about 2% (w/v) to about 12% (w/v), from about 3% (w/v) to about 11% (w/v) from about 4% (w/v) to about 10% (w/v), from about 5% (w/v) to about 9% (w/v), from about 6% (w/v) to about 8% (w/v), or about 7% (w/v). Preferably, trehalose is present in an amount of 2.5% (w/v) to 6% (w/v), such as 2.7% or such as 3.4%.

The incubating composition can comprise optional components, which can improve the ability to prepare freeze-dried cells that are viable upon rehydration. One optional component of the incubating composition is ethanol, which can be present in an amount of 0.1% to 5% (v/v), such as about 1%. Another optional component of the incubating composition is sorbitol, which can be present in an amount of 0.01% to 7.5% (v/v), such as about 3%. The incubating composition is preferably a buffered aqueous solution that includes at least a buffer, a salt, and a sugar, which in embodiments where a sugar is used as a cryoprotectant, is a different sugar than the cryoprotectant. In general, the components of the compositions herein are biologically tolerable at the concentrations used. For example, the buffer can be HEPES, bicarbonate, or another buffer or combination of buffers that is suitable for use in maintaining pH at a relatively neutral range, such as pH 6.2-7.8. In addition, the salt can be any biologically tolerable salt or combination of salts, where each salt or the combination is in the range of from about 3 mM to 150 mM, such as about 5 mM to 100 mM, about 5 mM to about 75 mM, or about 50 mM. Likewise, the sugar can be present in an amount ranging from about 2 mM to about 50 mM, such as from about 2 mM to about 20 mM, about 3 mM to about 10 mm, or about 5 mM. In an exemplary embodiment, the incubating buffer comprises: 9.5 mM HEPES, 75 mM NaCl, 4.8 mM KCl, 12 mM NaHCO₃, and 5 mM glucose (dextrose). In an exemplary embodiment, the incubating buffer comprises: 9.5 mM HEPES, 75 mM NaCl, 4.8 mM KCl, 12 mM NaHCO₃, and 3 mM glucose (dextrose). In general, the composition should be isotonic to the cells to avoid shrinking, swelling, or other deleterious effects on the cells.

In some embodiments, the incubating composition includes ethanol, which can be present in an amount of about 0.1% to 5% (v/v), about 0.5% (v/v) to 4.5% (v/v), about 1.0% (v/v) to about 4.0% (v/v), about 1.5% (v/v) to about 3.5% (v/v), about 2.0% (v/v) to about 3.0% (v/v), or about 2.5% (v/v).

In some embodiments, the incubating composition includes sorbitol, which can be present in an amount of about 0.01% to 5% (v/v), about 0.5% (v/v) to 4.5% (v/v), about 1.0% (v/v) to about 4.0% (v/v), about 1.5% (v/v) to about 3.5% (v/v), about 2.0% (v/v) to about 3.0% (v/v), or about 2.5% (v/v).

The freeze-dried PBMCs and rehydrated PBMCs produced from them can include one or more bioactive agents that can be introduced into the PBMCs prior to lyophilization, typically at the time of incubating the cells with cryoprotectant. While the bioactive agents generally may not contribute to cryoprotection or other aspects of production of the freeze-dried PBMCs they can be included for other purposes. One class of bioactive agents contemplated by the methods and compositions described herein are therapeutic substances, such as those generally referred to as drugs. These substances are typically released by the PBMCs upon rehydration and used in vivo and in vitro. Agents that are of a sufficiently small size to be taken up by the PBMCs during the incubating process are suitable for use in the methods and compositions described herein. Among the numerous bioactive agents useful in the methods and compositions described herein, non-limiting examples include antimicrobial agents (e.g., antibiotics, antivirals, antifungals), growth factors, anti-apoptotic agents, chemotherapeutic agents, antimitotic agents, hormones, and anti-toxins. While not being limited to any particular mode of action, it is presumed that the bioactive agents are taken up via the same process as the cryoprotectant. In some embodiments, the co-incubating of bioactive agents with cryoprotectant is not a required feature of the methods and compositions described herein, but instead provides additional advantages to the freeze-dried PBMCs and rehydrated freeze-dried PBMCs.

Furthermore, the freeze-dried PBMCs and rehydrated PBMCs produced from them can include one or more labeling agents or other markers for cells or biochemical activity. As with the bioactive agents discussed above, the labeling agents/markers can be introduced into the PBMCs prior to lyophilization, such as at the time of incubating the PBMCs with cryoprotectant. Non-limiting examples of labeling agents/markers are fluorescein, BODIPY, and ICG.

Nucleated cells that can be freeze-dried in methods provided herein can be genetically-modified nucleated cells. Although this specification and the Examples herein focus on PBMCs, it will be understood that the methods herein, for example for preparing freeze-dried PBMCs, can be used for any type of nucleated cells, not just PBMCs. For example, the methods can be applied to other nucleated blood cells, in some embodiments, for example, granulocytes, which in further embodiments can be neutrophils. In other embodiments, the nucleated cells are nucleated stem cells. Such stem cells can include, for example, adult stem cells, embryonic stem cells, or induced pluripotent stem cells (iPSCs). Preserved, and in illustrative embodiments freeze-dried, adult stem cells that can be included or produced using methods herein, include, but are not limited to hematopoietic stem cells (i.e. blood stem cells), hematopoietic progenitor cells (HPCs), and CD34+ cells, mesenchymal stem cells, neural stem cells, epithelial stem cells, or skin stem cells. In certain embodiments, the stem cells are amniotic membrane-derived mesenchymal stem cells (AMSCs). It will be understood in embodiments that include PBMC cell culture media, that cell culture media that is effective at culturing a particular nucleated cell type could be used instead, in the methods of preparing freeze-dried nucleated cells herein.

In addition to incubating the PBMCs with a cryoprotectant, the process of making freeze-dried PBMCs includes the use of an excipient or bulking agent in the lyophilization step. In some embodiments, the process includes contacting the incubated PBMCs with an excipient or bulking agent to create a second mixture (e.g., a lyophilization mixture) or the excipient or bulking agent as part of a mixture to which PBMCs are added to create a suspension that is lyophilized. It can, in some embodiments, also include contacting the incubated PBMCs with one or more additional proteins to create a lyophilization mixture. The excipient/bulking agent is added such that its final concentration in the lyophilization mixture is from about 0.1% to about 25% (w/v), such as from about 0.1% (w/v) to about 17% (w/v), such as from about 0.1% (w/v) to about 13% (w/v), such as from about 0.1% (w/v) to about 10% (w/v), such as between 1% and 10%, between 2.5% and 7.5%, or about 5%-6%. Excipients/bulking agents useful in the lyophilization mixture, include but are not limited to, polysucrose (e.g., Polysucrose™ 400), polyvinylpyrrolidone such as polyvinylpyrrolidone 40, maltose, and albumin (e.g., bovine serum albumin). Other excipients or bulking agents such as those known in the art can be used. In some embodiments, the polysucrose has a molecular weight (mw) ranging from about 300,000 mw to about 550,000 mw, such as about 400,000 mw. Preferably, Polysucrose400™ is used at a final concentration of 6%. When included, the proteins used can be any suitable protein. Alternatively or additionally, albumin, such as BSA or HSA is present.

In some embodiments, the process of making freeze-dried PBMCs includes contacting the PBMCs with an excipient or bulking agent such as trehalose or sorbitol, and optionally an alcohol (e.g., ethanol) at the same time to create a first mixture. In some embodiments, the process of making freeze-dried PBMCs includes contacting the PBMCs with an excipient or bulking agent such as trehalose, and optionally an alcohol (e.g., ethanol), after a first mixture is created to create a second mixture. In some embodiments, more than one excipient or bulking agent is added to make freeze-dried PBMCs. For example, a first excipient or bulking agent (e.g., any of the excipient or bulking agents described herein) can be added at the same time as trehalose and, optionally an alcohol (e.g., ethanol) and a second excipient or bulking agent (e.g., any of the excipient or bulking agents described herein) can be added at a later time to create a second mixture.

During or shortly after addition of the substances of the lyophilization mixture the PBMCs can be aliquoted according to the supplier's instructions. In some embodiments, the PBMC count is from about 10,000/4, to about 500,000/μL, such as from about 50,000/4, to about 300,000/μL, such as from about 150,000/4, to about 250,000/μL. Exemplary fresh cells are described in www.stemexpress.com/pub/media/productattachments/files/PB-MNC_PIS-F_RevG_0619.pdf, incorporated by reference in its entirety. In some embodiments, the concentration of the PBMCs in any one serum vial or other appropriate lyophilization vessel standard in the art is about the same concentration of PBMCs in another serum vial or other appropriate lyophilization vessel. The lyophilization vessels can be loosely stoppered, and placed into a lyophilization chamber.

The process of making freeze-dried nucleated cells further includes lyophilizing the lyophilization mixture. Samples can be lyophilized according to the following parameters.

Freezing is performed between −40° C. and −90° C. for one to six hours, after which primary drying is carried out below the glass transition temperature (Tg) point of the material. Typically, this requires drying at a temperature between −30° C. and −50° C. for about 5 to 15 hours, preferably about 10 hours. Secondary drying is then carried out above the Tg, such as between 10° C. and 40° C., preferably between 25° C. and 30° C., for about 3 to 10 hours, preferably about 5 hours. The cells are then held under vacuum at between 20° C. and 30° C. until removed from the lyophilizer. Table A shows exemplary lyophilization cycle conditions

TABLE A
Exemplary Lyophilization Conditions

| | | | Step Type |
|---|---|---|---|
| | Temp (° C.) | Time (min) | (pre-chilled) |
| | −40° C. | 180 | freeze hold |
| | Vacuum Pull (<100 mTorr) | | |
| Primary Dry | −30 | 120 | Ramp |
| | −30 | 1440 | Hold |
| | −20 | 120 | Ramp |
| | −20 | 1440 | Hold |
| | −10 | 120 | Ramp |
| | −10 | 120 | Hold |
| | −5 | 60 | Ramp |
| | −5 | 120 | Hold |
| Secondary Dry | (+) 25 | 300 | Ramp |
| | (+) 25 | >720 | Hold |

Once lyophilization is complete, the vessels/containers/vials are stoppered under a vacuum of less than 200 mTorr and then removed from the lyophilizer. Optionally, the stoppered vessels can be heat-treated at a temperature between 60° C. and 85° C. for about 12 to 36 hours. Where post-lyophilization heat treatment is used, it in certain embodiments that treatment is at 80° C. for 15 to 24 hours. However, in certain embodiments, freeze-dried PBMCs are not heat treated. In some embodiments, a process for preparing a PBMC composition, or a PBMC composition prepared by a process for preparing a PBMC composition as described herein, the process does not comprise heating a PBMC composition comprising freeze-dried PBMCs in the form of a solid to a temperature of 50° C. or above for more than 30 minutes. In some illustrative embodiments, the process does not comprise heating a PBMC composition comprising freeze-dried PBMCs in the form of a solid to a temperature of 60° C. or higher, for example, 65° C., 70° C., 75° C., 80° C., 85° C., 90° C., 95° C., 100° C., or higher, for more than 1, 2, 3, 4, 5, 10, 15, 20, 30 or 45 minutes, or for more than 1, 2, 3, 4, 5, 6, 7, or 8 hours. In illustrative embodiments, the methods herein do not comprise heating the PBMC composition comprising freeze-dried PBMCs in the form of a solid to a temperature of 60° C. or above for more than 1 hour.

The methods and compositions described herein also provide a process for preparing rehydrated PBMCs. In brief, the process includes contacting the freeze-dried PBMCs of the methods and compositions described herein with an aqueous composition under conditions where the freeze-dried PBMCs internalize at least the water of the composition to cause rehydration of the PBMCs. The step of contacting can be any action that results in the water coming into physical contact with the freeze-dried PBMCs and being taken into the PBMCs to rehydrate them. In preferred embodiments, an aqueous composition is added to the vessel containing the freeze-dried PBMCs to effect rehydration. The PBMCs are then allowed to rehydrate. If desired, gentle agitation of the vessel can be performed to separate the dried PBMCs and accelerate rehydration of the PBMCs. The aqueous composition can include, in addition to water, any number of additional components suitable for maintenance of PBMCs in a viable state. Such components, are not only limited to only the components listed herein. For example, freeze-dried PBMCs sample can be rehydrated with water, a PBMC cell culture media, or a water/buffer/plasma mixture. In some embodiments, the PBMCs are rehydrated in a volume of water that is equal to the volume of the lyophilization mixture added to the vial before lyophilization.

As discussed in more detail herein, another aspect of the methods and compositions described herein, relate to populations of freeze-dried PBMCs and populations of rehydrated freeze-dried PBMCs. Freezing PBMCs can be performed using various processes. For example, when freezing PBMCs processes that include DMSO, glycerol, ethylene glycol, hydroxyethyl starch, and any combination thereof have been used (See, e.g., Mallone, R., Isolation and preservation of peripheral blood mononuclear cells for analysis of islet antigen-reactive T cell responses: position statement of the T-Cell Workshop Committee of the Immunology of Diabetes Society, Clinical and Experimental Immunology, 163: 33-49 (2010), which is incorporated herein by reference in its entirety). Populations according to the methods and compositions described herein, have a percentage or proportion of viable PBMCs. Cell viability levels in populations according to the methods and compositions described herein, such as for example, Trypan Blue staining can reach about 20.0%.

After lyophilization of the PBMCs various assays can be used to assess the viability of the cells. For example, colorimetric assays, such as Trypan Blue staining and MTT (3-(4,5-dimethylthiazol-2-yl)-2,5-diphenyltetrazolium bromide) assay are two exemplary methods for assessing viability of cells, but suitable methods that could be employed by a person skilled in the art are not limited to these methods.

For example, Trypan Blue staining assesses a cell's ability to exclude a dye. For example, if the lipid bilayer cell membrane remains intact and the cell, the cell has the ability to exclude the dye which is indicative of cell viability. The MTT assay assesses a cell's ability to metabolize, grow and divide, and thus can be used as a marker for viable cells and as an assay for viable cell metabolic activity. More specifically, the MTT assay is a direct measurement of oxidoreductase enzymatic activity, which is an indicator of cell viability/metabolic activity and proliferation. Some embodiments herein are methods for preserving and/or enhancing % max activity of a population of PBMCs, which can also be referred to as methods for preserving and/or enhancing metabolic activity of PBMCs, as discussed in more detail herein.

More specific, non-limiting aspects and embodiments of methods of preparing freeze-dried PBMCs include the following:

In one aspect, provided herein is a method of preparing freeze-dried peripheral blood mononuclear cells PBMCs, the method comprising:
A) incubating PBMCs, in some embodiments at a temperature between 27° C. and 42° C., 32° C. and 42° C., or 35° C. and 39° C., or at about or exactly 37° C., in certain embodiments for between 5 minutes and 8 hours, for example between 10 minutes and 2 hours, suspended in a mixture comprising polysucrose, trehalose, sorbitol, and albumin in an aqueous environment to form incubated PBMCs suspended in the mixture; and
B) lyophilizing the incubated PBMCs suspended in the mixture to form a solid composition comprising freeze-dried PBMCs. Such solid composition is typically a powder with less than 1% water content.

In another aspect, provided herein is a method of preparing freeze-dried peripheral blood mononuclear cells (PBMCs), the method comprising: A. incubating PBMCs at a temperature of between 35° C. and 40° C. for between 10 minutes and 120 minutes, in an aqueous mixture to form incubated PBMCs suspended in the mixture, wherein the mixture comprises i) trehalose, wherein the trehalose is present in the aqueous mixture at a concentration of from 0.1% (w/v) to 10.0% (w/v); ii) an excipient comprising polysucrose and/or albumin, wherein the polysucrose and/or the albumin are present in the aqueous medium, at a concentration individually or in combination from 4.0% (w/v) to 20.0% (w/v); iii) a buffer; and either one or both iv) sorbitol and PBMC cell culture media components, wherein the sorbitol if present, is present in the aqueous medium at a concentration from 0.1% (w/v) to 5.0% (v/v), and the cell culture media components if present comprise amino acids, vitamins, and inorganic salts, at effective concentrations to facilitate culturing of PBMCs; and B. lyophilizing the incubated PBMCs suspended in the mixture to form a solid composition comprising freeze-dried PBMCs.

In one aspect of preparing freeze-dried peripheral blood mononuclear cells (PBMCs) the aqueous mixture comprises a PBMC cell culture media and a buffer that comprises the buffer of the cell culture media.

In further aspects of preparing freeze-dried peripheral blood mononuclear cells (PBMCs), the method comprising: A. incubating between 1×10⁶ and 1×10¹² freeze-dried PBMCs at a temperature of between 35 C and 40 C for between 10 minutes and 120 minutes, in an aqueous medium comprising trehalose at a concentration of from 0.1% (w/v) to 10.0% (w/v), an excipient comprising polysucrose and/or albumin at a concentration from 0.1% (w/v) to 15% (w/v), and DMSO at a concentration of from 2% to 5%, to form incubated PBMCs suspended in the mixture; and B. lyophilizing the incubated PBMCs suspended in the mixture to form a solid composition comprising freeze-dried PBMCs.

In some embodiments of a method of preparing a PBMC composition, the method comprises isolating PBMCs using a method that typically includes collecting blood in a vessel comprising an anti-coagulant. In some embodiments, the anti-coagulant can comprise any well-known anti-coagulant known to process the blood from which PBMCs have to be isolated. However, in some embodiments, a method for preparing a PBMC composition comprises isolating PBMCs using a method that does not comprise heparin-treated blood.

In some embodiments, a PBMC composition as described herein does not comprise fibrinogen. In some embodiments, a process for preparing a PBMC composition, or a PBMC composition prepared by a process for preparing a PBMC composition as described herein, the process at any step does not comprise contacting any of the components during the process with fibrinogen.

Further exemplary aspects and embodiments of processes for preparing freeze-dried PBMCs are provided throughout this specification. As an example, such processes/methods are provided in the Exemplary Embodiments section.

Methods of Preserving or Enhancing Metabolic Activity

It will be understood, as demonstrated in the Examples section herein, for example using the MTT assay, that processes/methods for preparing freeze-dried nucleated cells (e.g. PBMCs), in certain embodiments, result in populations of freeze-dried nucleated cells (e.g. PBMCs) wherein at least some of the nucleated cells (e.g. PBMCs) in the population have preserved or even enhanced metabolic activity when such population of freeze-dried nucleated cells (e.g. PBMCs) are tested after rehydration. Thus, it will be understood that methods for preparing freeze-dried nucleated cells (e.g. PBMCs) herein, in some aspects are methods for preparing freeze-dried nucleated cells (e.g. PBMCs) with preserved or enhanced metabolic activity.

Thus, provided herein in some embodiments and aspects provided herein are processes/methods for preparing freeze-dried (e.g., lyophilized) PBMCs and for preparing rehydrated PBMCs that had been freeze-dried, having preserved or enhanced metabolic activity. The processes/methods typically include the following steps:

A) incubating PBMCs in a liquid, typically an aqueous solution that includes a cryoprotectant, which in illustrative embodiments is trehalose and/or DMSO, and a lyoprotectant, that in illustrative embodiments is polysucrose, DMSO, and/or albumin; and
B) lyophilizing the liquid to form freeze-dried PBMCs.

In addition to benefits with freeze-dried PBMCs, it will be understood, as demonstrated in the Examples section herein, for example using the MTT assay, that certain formulations herein are well-suited for incubating and/or culturing PBMCs even without freeze-drying the PBMCs. Incubation or culturing in the formulations can result in a population PBMCs with enhanced growth and/or metabolic activity. Thus, it will be understood that any of the mixtures provided herein that include PBMCs, are separate aspects, and in some aspects are improved PBMC media formulations. For example, in one aspect provided herein is an aqueous mixture comprising a population of PBMCs suspended in any of the aqueous mixtures provided herein. In illustrative embodiments, such aqueous mixtures comprise some, most, or all components of a PBMC cell culture media, such as a buffer, amino acids, vitamins, and sugars, as well as trehalose, sorbitol, BSA, and/or polysucrose. Specific components and concentrations thereof, of these various components in illustrative embodiments are any of the component mixtures and concentration ranges of these components, provided herein in methods of preparing freeze-dried PBMCs. These mixtures themselves are enhanced PBMC formulations. And PBMCs suspended in such mixtures are further aspects provided herein. In these aspects, such suspended PBMCs, or one or more cell types therein, can be used for any purpose that PBMCs and/or such cell type(s) are used and are not necessarily subjected to a lyophilization or other freeze-drying procedure. Some embodiments of such aspects of mixtures/formulations and PBMCs suspended in such mixtures/formulations comprise components and concentrations within 20%, 15%, 10%, 5%, 4%, 3%, 2%, of 1% of those components found in Formula 5, and in illustrative embodiments, Formula 6 herein. In some embodiments, such formulation used in these embodiments with suspended PBMCs that are not freeze-dried, comprises an RPMI media supplemented with additional components that include trehalose, sorbitol, BSA, and/or polysucrose, at any of the concentrations/ranges provided herein for compositions that include freeze-dried PBMCs for such additional components.

Thus, in one aspect, provided herein is a method of preserving or enhancing % max activity, or a method of preserving or enhancing metabolic activity of a population of PBMCs, in certain embodiments freeze-dried PBMCs, the method comprising: incubating the population of PBMCs with a mixture comprising trehalose, sorbitol, BSA, and optionally a bulking agent in an aqueous environment to form a population of PBMCs with enhanced % max activity, or with preserved or enhanced metabolic activity, respectively. Further aspects and embodiments related to % max activity and metabolic activity can be found in this disclosure, including, but not limited to the Exemplary Embodiments section herein.

For example, methods of enhancing % max activity, or a method of enhancing metabolic activity of a population of PBMCs (e.g., fresh PBMCs) include incubating the population of fresh PBMCs in a solution (e.g., buffer) derived from rehydrated PBMCs. In some embodiments, PBMCs lyophilized in any of the lyophilization formulations described herein (e.g., formulations 1-6) are rehydrated. In some embodiments, PBMCs lyophilized in formulation 3, formulation 5, or formulation 6 are rehydrated. In some embodiments, the rehydrated PBMCs are removed by centrifugation and/or filtration. In such embodiments, the resulting solution after centrifugation and/or filtration, when incubated with a population of fresh PBMCs, results in enhanced % max activity of the population of PBMCs (e.g., fresh PBMCs).

As used herein, “max activity” is the metabolic and mitochondrial activity of fresh PBMCs as measured by MTT. The “% max activity” referred to herein is the activity of a PBMC sample that is normalized to the activity of fresh PBMC samples, as measured, for example, by OD (optical density) values. In other words, the % max activity is the relative metabolic activity as compared to fresh PBMCs.

In some embodiments of preparing a population of PBMCs, in illustrative embodiments preserved (e.g. freeze-dried) PBMCs, by any of the methods described herein, the population has a % max activity of at least about 15%. In some embodiments of preparing a population of preserved (e.g. freeze-dried) PBMCs, in illustrative embodiments preserved (e.g. freeze-dried) PBMCs, by any of the methods described herein, the population has a % max activity of at least about 20%. In some embodiments of preparing a population of PBMCs, in illustrative embodiments preserved (e.g. freeze-dried) PBMCs, by any of the methods described herein, the population has a % max activity of at least about 25%. In some embodiments of preparing a population of PBMCs, in illustrative embodiments preserved (e.g. freeze-dried) PBMCs, by any of the methods described herein, the population has a % max activity of at least about 30%. In some embodiments of preparing a population of PBMCs, in illustrative embodiments preserved (e.g. freeze-dried) PBMCs, by any of the methods described herein, the population has a % max activity of at least about 35%. In some embodiments of preparing a population of PBMCs, in illustrative embodiments preserved (e.g. freeze-dried) PBMCs, by any of the methods described herein, the population has a % max activity of at least about 40%.

In some embodiments of preparing a population of PBMCs, in illustrative embodiments preserved (e.g. freeze-dried) PBMCs, by any of the methods described herein, the population has a metabolic activity that is between 15% and 40% of the metabolic activity of fresh PBMCs.

In some embodiments, % max activity, such as the % max activity of a population of PBMCs, in illustrative embodiments preserved (e.g. freeze-dried) PBMCs, is % max activity as determined by a colorimetric assay. In some embodiments, % max activity is % max activity as determined by MTT (3-(4,5-dimethylthiazol-2-yl)-2,5-diphenyltetrazolium bromide) assay.

In some embodiments, a population of PBMCs, in illustrative embodiments lyophilized PBMCs in compositions and methods herein can have a maximum (“max”) activity as measured by a colorimetric assay (e.g., MTT assay) of at least about 1% to at least about 40% max activity relative to PBMCs in RPMI media. For example, lyophilized PBMCs can have a max activity as measured by the MTT assay of at least about 5% to at least about 35%, at least about 10% to at least about 30%, at least about 15% to at least about 25%, at least about 20% max activity relative to PBMCs in RPMI media. In some embodiments, lyophilized PBMCs can have a % max activity as measured by the MTT assay of at least about at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, at least about 10%, at least about 10.5%, at least about 11%, at least about 11.5%, at least about 12%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 20.5%, at least about 21%, at least about 21.5%, at least about 22%, at least about 22.5%, at least about 23%, at least about 23.5%, at least about 24%, at least about 24.5%, at least about 25%, at least about 25.5%, at least about 26%, at least about 26.5%, at least about 27%, at least about 27.5%, at least about 28%, at least about 28.5%, at least about 29%, at least about 29.5%, at least about 30%, at least about 30.5%, at least about 31%, at least about 31.5%, at least about 32%, at least about 32.5%, at least about 33%, at least about 33.5%, at least about 34%, at least about 34.5%, at least about 35%, at least about 35.5%, at least about 36%, at least about 36.5%, at least about 37%, at least about 37.5%, at least about 38%, at least about 38.5%, at least about 39%, at least about 39.5% or at least about 40% max activity relative to PBMCs in RPMI media.

As another non-limiting aspect, provided herein is a method of preserving metabolic activity of a population of freeze-dried peripheral blood mononuclear cells PBMCs the method comprising:

A) incubating PBMCs, in some embodiments at a temperature between 20° C. and 40° C., 25° C. and 40° C., 27° C. and 42° C., 32° C. and 42° C., or 35° C. and 39° C., or at about or exactly 37° C., in certain embodiments for between 5 minutes and 8 hours, for example between 10 minutes and 2 hours, between 30 minutes and 2 hours, suspended in a mixture comprising polysucrose, trehalose, sorbitol, and albumin in an aqueous environment to form incubated PBMCs suspended in the mixture; and

B) lyophilizing the incubated PBMCs suspended in the mixture to form a solid composition comprising freeze-dried PBMCs. Such solid composition is typically a powder with less than 1% water content.

In another aspect, provided herein is a method of preserving metabolic activity of a population of freeze-dried peripheral blood mononuclear cells (PBMCs), the method comprising:

A. incubating PBMCs at a temperature of between 35° C. and 40° C. for between 10 minutes and 120 minutes, in an aqueous mixture to form incubated PBMCs suspended in the mixture, wherein the mixture comprises i) trehalose, wherein the trehalose is present in the aqueous mixture at a concentration of from 0.1% (w/v) to 10.0% (w/v); ii) an excipient comprising polysucrose and/or albumin, wherein the polysucrose and/or the albumin are present in the aqueous medium, at a concentration individually or in combination from 4.0% (w/v) to 20.0% (w/v); iii) a buffer; and either one or both iv) sorbitol and PBMC cell culture media components, wherein the sorbitol if present, is present in the aqueous medium at a concentration from 0.1% (w/v) to 5.0% (v/v), and the cell culture media components if present comprise amino acids, vitamins, and inorganic salts, at effective concentrations to facilitate culturing of PBMCs; and B. lyophilizing the incubated PBMCs suspended in the mixture to form a solid composition comprising freeze-dried PBMCs.

Further aspects and embodiments of preserving metabolic activity of a population of freeze-dried PBMCs are provided in other sections herein, for example, but not limited to the Exemplary Embodiments section. Such methods include any of the methods for preparing freeze-dried PBMCs provided herein.

Thus, in some embodiments, provided herein is a method of preserving % max activity or a method of preserving metabolic activity of a population of PBMCs, the method comprising:

incubating the population of PBMCs with a first mixture comprising trehalose, sorbitol, and BSA in an aqueous environment to form incubated PBMCs; and
contacting the incubated PBMCs with polysucrose to form a population of PBMCs with enhanced preserved % max activity or metabolic activity, respectively.

Thus, provided herein, is a method of enhancing % max activity or a method of enhancing metabolic activity of a population of PBMCs, the method comprising: contacting PBMCs with a mixture comprising trehalose, sorbitol, BSA, and polysucrose in an aqueous environment to form a population of PBMCs with enhanced % max activity or enhanced metabolic activity, respectively.

Thus, provided herein, is a method of enhancing % max activity or a method of enhancing metabolic activity of a population of PBMCs, the method comprising: incubating the population of PBMCs with a mixture comprising polysucrose, trehalose, and DMSO in an aqueous environment to form a population of PBMCs with enhanced % max activity, or enhanced metabolic activity, respectively.

Thus, provided herein, is a method of enhancing % max activity of a population of PBMCs or a method of enhancing metabolic activity, the method comprising: contacting PBMCs with a mixture comprising trehalose and BSA in an aqueous environment to form a population of PBMCs with enhanced % max activity, or enhanced metabolic activity, respectively.

Thus, provided herein, is a method of enhancing % max activity, or a method of enhancing metabolic activity of a population of PBMCs, the method comprising: incubating the population of PBMCs with a mixture comprising trehalose and BSA in an aqueous environment to form a population of PBMCs with enhanced % max activity, or enhanced metabolic activity, respectively.

Methods of Preserving Viability of a Population of PBMCs

It will be understood that methods of preparing freeze-dried nucleated cells, in illustrative embodiments PBMCs, provided herein prepare populations of freeze-dried nucleated cells (e.g. PBMCs) in which some of the freeze-dried cells in the population are viable. Thus, it will be understood that methods for preparing freeze-dried nucleated cells (e.g. PBMCs) herein, in some aspects are methods for preserving nucleated cells (e.g. PBMCs), or methods for preserving viability of freeze-dried nucleated cells (e.g. PBMCs).

In some embodiments and aspects provided herein are processes/methods for preserving viability in a population of PBMCs, such as freeze-dried (e.g., lyophilized) PBMCs and for preparing a population of rehydrated PBMCs with preserved viability. The processes/methods typically include the following steps:

A) incubating PBMCs in a liquid, typically an aqueous solution that includes a cryoprotectant, which in illustrative embodiments is trehalose and/or DMSO, and a lyoprotectant, that in illustrative embodiments is polysucrose, DMSO, and/or albumin; and
B) lyophilizing the liquid to form freeze-dried PBMCs.

In some embodiments, provided herein are methods of preserving viability of a population of PBMCs, that include incubating the population of PBMCs with a mixture comprising, trehalose, sorbitol, BSA, and optionally a bulking agent in an aqueous environment to form incubated PBMCs and lyophilizing the mixture to form a population of incubated PBMCs with preserved viability.

Thus, in some embodiments, provided herein is a method of preserving viability of a population of PBMCs, the method comprising:

incubating the population of PBMCs with a first mixture comprising trehalose, sorbitol, and BSA in an aqueous environment to form incubated PBMCs; and contacting the incubated PBMCs with polysucrose.

Thus, in some embodiments, provided herein is a method of preserving viability of a population of PBMCs, the method comprising:

incubating the population of PBMCs with a mixture comprising trehalose, sorbitol, BSA, and polysucrose in an aqueous environment to form incubated PBMCs.

Thus, in some embodiments, provided herein is a method of preserving viability of a population of PBMCs, the method comprising:

contacting PBMCs with a mixture comprising trehalose, sorbitol, BSA, and polysucrose in an aqueous environment to form a population of incubated PBMCs with preserved viability; and lyophilizing the mixture to form a population of incubated PBMCs with preserved viability.

Thus, in some embodiments, provided herein is a method of preserving viability of a population of PBMCs, the method comprising:

incubating the population of PBMCs with a mixture comprising, trehalose, sorbitol, and BSA in an aqueous environment to form incubated PBMCs;
contacting the incubated PBMCs with polysucrose; and lyophilizing the mixture to form a population of incubated PBMCs with preserved viability.

Thus, provided herein is a method of preserving viability of a population of PBMCs, the method comprising: contacting PBMCs with a mixture comprising polysucrose, trehalose, and DMSO in an aqueous environment to form a population of incubated PBMCs with preserved viability; and lyophilizing the mixture to form a population of incubated PBMCs with preserved viability.

Thus, provided herein is a method of preserving viability of a population of PBMCs, the method comprising: incubating the population of PBMCs with a mixture comprising polysucrose, trehalose, and DMSO in an aqueous environment to form incubated PBMCs; and lyophilizing the mixture to form a population of incubated PBMCs with preserved viability.

Thus, provided herein, is a method of preserving viability of a population of PBMCs, the method comprising: contacting PBMCs with a mixture comprising trehalose and BSA in an aqueous environment to form a population of incubated PBMCs with preserved viability; and lyophilizing the mixture to form a population of incubated PBMCs with preserved viability.

Thus, provided herein, is a method of preserving viability of a population of PBMCs, the method comprising: incubating the population of PBMCs with a mixture comprising trehalose and BSA in an aqueous environment to form incubated PBMCs; and lyophilizing the mixture to form a population of incubated PBMCs with preserved viability.

In some embodiments of preparing a population of preserved PBMCs by any of the methods described herein, the method comprises lyophilizing the population of PBMCs.

As another non-limiting aspect, provided herein is a method of preserving viability of a population of freeze-dried peripheral blood mononuclear cells PBMCs the method comprising:

A) incubating PBMCs, in some embodiments at a temperature between 20° C. and 40° C., 25° C. and 40° C., 27° C. and 42° C., 32° C. and 42° C., or 35° C. and 39° C., or at about or exactly 37° C., in certain embodiments for between 5 minutes and 8 hours, for example between 10 minutes and 2 hours, between 30 minutes and 2 hours, suspended in a mixture comprising polysucrose, trehalose, sorbitol, and albumin in an aqueous environment to form incubated PBMCs suspended in the mixture; and

B) lyophilizing the incubated PBMCs suspended in the mixture to form a solid composition comprising freeze-dried PBMCs. Such solid composition is typically a powder with less than 1% water content.

In another aspect, provided herein is a method of preserving viability of a population of freeze-dried peripheral blood mononuclear cells (PBMCs), the method comprising:

A. incubating PBMCs at a temperature of between 35° C. and 40° C. for between 10 minutes and 120 minutes, in an aqueous mixture to form incubated PBMCs suspended in the mixture, wherein the mixture comprises i) trehalose, wherein the trehalose is present in the aqueous mixture at a concentration of from 0.1% (w/v) to 10.0% (w/v); ii) an excipient comprising polysucrose and/or albumin, wherein the polysucrose and/or the albumin are present in the aqueous medium, at a concentration individually or in combination from 4.0% (w/v) to 20.0% (w/v); iii) a buffer; and either one or both iv) sorbitol and PBMC cell culture media components, wherein the sorbitol if present, is present in the aqueous medium at a concentration from 0.1% (w/v) to 5.0% (v/v), and the cell culture media components if present comprise amino acids, vitamins, and inorganic salts, at effective concentrations to facilitate culturing of PBMCs; and

B. lyophilizing the incubated PBMCs suspended in the mixture to form a solid composition comprising freeze-dried PBMCs.

Further aspects and embodiments of preserving viability of a population of freeze-dried PBMCs are provided in other sections herein, for example, but not limited to the Exemplary Embodiments section. Such methods include any of the methods for preparing freeze-dried PBMCs provided herein.

In some embodiments of preparing a population of PBMCs by any of the methods described herein, the PBMCs are mammalian PBMCs. In some embodiments of preparing a population of PBMCs by any of the methods described herein, the PBMCs are human PBMCs. In some embodiments of preparing a population of PBMCs by any of the methods described herein, the PBMCs are lymphocytes, monocytes, or granulocytes. In some embodiments of preparing a population of PBMCs by any of the methods described herein, the lymphocytes are B-cells or T-cells.

Thus, provided herein, are methods of preparing freeze-dried PBMCs, the method comprising:

incubating PBMCs with a mixture comprising polysucrose, trehalose, sorbitol, and BSA in an aqueous environment to form incubated PBMCs; and lyophilizing the mixture to form freeze-dried PBMCs.

Thus, provided herein, are freeze-dried PBMCs prepared by a process comprising the steps of:

incubating PBMCs with a mixture comprising polysucrose, trehalose, sorbitol, and BSA in an aqueous environment to form incubated PBMCs; and lyophilizing the mixture, to form freeze-dried PBMCs.

In some embodiments, viability, such as the viability of a population of PBMCs, is viability as determined by a colorimetric assay. In some embodiments, viability is viability as determined by Trypan Blue staining.

After lyophilization of the PBMCs various assays can be used to assess the viability of the cells. For example, dye staining assays, such as Trypan Blue staining can be used for assessing viability of cells, although other methods are known to a person skilled in the art.

In some embodiments, lyophilized PBMCs described herein can have a viability as measured by a colorimetric assay (e.g., Trypan Blue staining) of about 0.5% to about 20%, such as for example about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%.

Viability levels can vary depending on the particular cells and parameters used. For example, in a colorimetric assay (e.g., Trypan Blue staining assay) viability levels can reach or exceed at least about 0.5%, at least about 1%, at least about 1.5%, at least about 2.0%, at least about 2.5%, at least about 3.0%, at least about 3.5%, at least about 4.0%, at least about 4.5%, at least about 5.0%, at least about 5.5%, at least about 6.0%, at least about 6.5%, at least about 7.0%, at least about 7.5%, at least about 8.0%, at least about 8.5%, at least about 9.0%, at least about 9.5%, at least about 10.0%, at least about 10.5%, at least about 11.0%, at least about 11.5%, at least about 12.0%, at least about 12.5%, at least about 13.0%, at least about 13.5%, at least about 14.0%, at least about 14.5%, at least about 15.0%, at least about 15.5%, at least about 16.0%, at least about 16.5%, at least about 17.0%, at least about 17.5%, at least about 18.0%, at least about 18.5%, at least about 19.0%, at least about 19.5%, at least about 20.0%, at least about 20%, at least about 20.5%, at least about 21%, at least about 21.5%, at least about 22%, at least about 22.5%, at least about 23%, at least about 23.5%, at least about 24%, at least about 24.5%, at least about 25%, at least about 25.5%, at least about 26%, at least about 26.5%, at least about 27%, at least about 27.5%, at least about 28%, at least about 28.5%, at least about 29%, at least about 29.5%, at least about 30%, at least about 30.5%, at least about 31%, at least about 31.5%, at least about 32%, at least about 32.5%, at least about 33%, at least about 33.5%, at least about 34%, at least about 34.5%, at least about 35%, at least about 35.5%, at least about 36%, at least about 36.5%, at least about 37%, at least about 37.5%, at least about 38%, at least about 38.5%, at least about 39%, at least about 39.5%, or at least about 40%.

Populations according to the methods and compositions described herein are typically in vitro compositions, such as a population of cells contained in a vessel, container, vial, syringe, etc., which are maintained or grown until used in in vivo or in vitro applications. The compositions typically contain, in addition to the PBMCs, an aqueous environment that is suitable for maintaining the PBMCs in a viable state until they are used for the various purposes that cell compositions are used, including those discussed herein.

In some embodiments, buffers (e.g., any of the buffers described herein) can be used to rehydrate lyophilized PBMCs. For example, a sample of freeze-dried (e.g., lyophilized) PBMCs can be rehydrated in a buffer, centrifuged, and filtered through a filter to remove rehydrated PBMCs (e.g., about 0.1 to about 1.0 μm filter).

In some embodiments, a buffer used to rehydrate lyophilized PBMCs is used to preserve PBMCs, e.g., fresh PBMCs. For example, buffer from rehydrated PBMCs can be used instead of media, such as for example, RPMI. In some embodiments, the buffer (e.g., any of the buffers described herein) from rehydrated and lyophilized PBMCs can preserve PBMCs at least about the same as RPMI media. In some embodiments, the buffer from rehydrated and filtered lyophilized PBMCs can preserve PBMCs at least about 1.0× to about 2.5× relative to RPMI media as measured by OD (590 nM) in the MTT assay.

Methods of Administering a PBMC Composition to a Subject

In some aspects, provided herein is a method for administering to a subject, a preserved nucleated cell (e.g. PBMC) composition comprising a population of preserved nucleated cells provided herein, which in illustrative embodiments is a rehydrated nucleated cell (e.g. PBMC) composition. The method typically includes administering to the subject, a therapeutically effective number of rehydrated nucleated cells. In related illustrative aspects with similar or the same steps, the method is a method for treating a disease or disorder. In some embodiments, the disease or disorder is a disease or disorder that can be treated by cell therapy, for example stem cell therapy, or a cell therapy that involves PBMCs, or a subset thereof. For example, such therapy can be CAR-T therapy. In some embodiments, the disease is cancer, or any other disorder that includes administering the preserved nucleated cells (e.g. PBMCs) to a subject. In some embodiments, the administering can include administering topically. Administering can include administering parenterally. Administering can include administering intravenously. Administering can include administering intramuscularly. Administering can include administering intrathecally. Administering can include administering subcutaneously. Administering can include administering intraperitoneally.

It would be understood that in certain embodiments, a PBMC composition as described herein in the form of a solid that is packaged in a vial/container can be rehydrated to a target volume before administering to the subject. In some embodiments, between 5 and 100 ml, 10 and 100 ml, 5 and 50 ml, and 10 and 50 ml of rehydrated preserved nucleated cell (e.g. freeze-dried PBMCs) are administered to the subject. The number of preserved nucleated cells (e.g. freeze-dried PBMCs) delivered in some embodiments, is between 1×10⁵, or 1×10⁶, or 1×10⁷ preserved nucleated cells (e.g. freeze-dried PBMCs) on the low end of the range, and 1×10, 1×10⁹, 1×10, 1×10¹¹, or 1×10¹² nucleated cells (e.g. freeze-dried PBMCs) on the high end of the range. The preserved nucleated cells (e.g. freeze-dried PBMCs) in some embodiments are allogeneic preserved nucleated cells (e.g. allogeneic freeze-dried PBMCs).

### EXEMPLARY EMBODIMENTS

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional aspects and embodiments are provided in other sections herein. Furthermore, it will be understood that embodiments are provided that are specific embodiments (e.g. sub-embodiments) that can be combined with any aspect or other embodiment, for example as discussed in this entire disclosure. It is intended in view of the full disclosure herein, that any individual embodiment recited below or in this full disclosure can be combined with any aspect or other embodiment recited below or in this full disclosure where it is an additional element that can be added to an aspect or another embodiment or because it is a narrower element (e.g. a sub-element) for an element already present in an aspect or another embodiment. Such combinations are sometimes provided as non-limiting exemplary combinations and/or are discussed more specifically in other sections of this detailed description.

It will be understood that some aspects and embodiments herein comprise compositions in a solid form, which can in non-limiting examples, be a powder. The same or related aspects and embodiments are compositions with having a water content less than 1%, 0.9%, 0.8%, 0.75%, 0.7%, 0.6% or 0.5% water, or between 0.01% and 1%, 0.9%, 0.8%, 0.75%, 0.7%, 0.6% or 0.5% water, or between 0.1% and 1%, 0.9%, 0.8%, 0.75%, 0.7%, 0.6% or 0.5% water. It will be understood that when a solid composition component recites “when rehydrated” or similar phrases, typically this refers to a property of the component of the solid composition or the composition itself. Thus, aspects and embodiments that recite “when rehydrated” are not intended to be product by process compositions unless such aspects or embodiments recite active method steps. Rather such “when rehydrated” or similar language is present to provide characteristics of components of the solid composition or of the solid composition itself. Provided herein in other aspects are liquid compositions that are rehydrated products that result when the solid compositions are rehydrated to a target volume. Such aspects or embodiments typically have an active explicit step of hydrating or rehydrating. Furthermore, typically, such a target rehydration volume is the volume of a composition that was dehydrated to form the solid composition that is being rehydrated. In illustrative embodiments liquid compositions with suspended PBMCs are freeze-dried to form solid, to obtain the solid (e.g. powder) compositions herein.

Provided herein in one aspect is a peripheral blood mononuclear cell (PBMC) composition in the form of a solid, comprising

A) a cryoprotectant, which in illustrative embodiments is or comprises trehalose, wherein in certain embodiments when the composition is rehydrated, the cryoprotectant (e.g. trehalose) is present at a concentration of from about or exactly 0.1% (w/v) to about or exactly 10.0% (w/v);

B) a lyoprotectant, which in illustrative embodiments is one, two or all of polysucrose albumin, and sorbitol, wherein in certain embodiments when the composition is rehydrated the polysucrose, albumin, and sorbitol are each, both or all in combination, present in the aqueous environment at a concentration from about or exactly 0.1% (w/v) to about or exactly 25% (w/v); and

C) a population of freeze-dried peripheral blood mononuclear cells (PBMCs), that in some embodiments is between 1×10⁵ and 1×10¹¹ freeze-dried PBMCs. In certain embodiments when the composition is rehydrated, between about or exactly 1% and 25% of the PBMCs in said population are viable. In certain embodiments either i) the composition further comprises DMSO, which in certain embodiments when the composition is rehydrated is present at a concentration between exactly or about 1% and exactly or about 5%; or ii) wherein in embodiments where sorbitol is not present, the composition further comprises PBMC cell culture media components, which in illustrative embodiments includes amino acids, vitamins, and inorganic salts. In certain embodiments, the PBMC cell culture media or the composition otherwise can further include a buffer (or the cell culture media can include an additional buffer if another buffer is present in the composition). In certain embodiments the composition when rehydrated to a target volume comprises the PBMC cell culture media components at concentrations that effectively support PBMC cell culturing and in certain most illustrative embodiments are the concentrations that are intended for such media when used for PBMC culturing.

In certain aspects, provided herein is a peripheral blood mononuclear cell (PBMC) composition in liquid form, comprising

A) a cryoprotectant, which in illustrative embodiments is or comprises trehalose, wherein in certain embodiments the cryoprotectant (e.g. trehalose) is present in the aqueous environment at a concentration of from about or exactly 0.1% (w/v) to about or exactly 10.0% (w/v);

B) a lyoprotectant, which in illustrative embodiments is one, two or all of polysucrose albumin, and sorbitol, wherein in certain embodiments the polysucrose, albumin, and sorbitol are each, both or all in combination, present at a concentration from about or exactly 0.1% (w/v) to about or exactly 25% (w/v);

and

C) a population of freeze-dried peripheral blood mononuclear cells (PBMCs) suspended therein, that in some embodiments is between 1×10⁵ and 1×10¹¹ freeze-dried PBMCs.

In certain embodiments between about or exactly 1% and 25% of the PBMCs in said population are viable.

In certain embodiments

i) either the composition further comprises DMSO, which in certain embodiments is present at a concentration between exactly or about 1% and exactly or about 5%; or

ii) wherein in embodiments where sorbitol is not present, the composition further comprises PBMC cell culture media components. In certain illustrative embodiments the PBMC cell culture media components include amino acids, vitamins, and inorganic salts, and in certain embodiments can further include a buffer (or an additional buffer if another buffer is otherwise present in the composition). In certain embodiments the composition comprises the PBMC cell culture media components at concentrations that effectively support PBMC cell culturing. In certain most illustrative embodiments the PBMC cell culture media components are at concentrations that are intended for such media when used for PBMC culturing and are effective for culturing PBMCs.

In certain illustrative embodiments, the composition comprises both the sorbitol and the PBMC cell culture media components.

In one aspect, provided herein is a peripheral blood mononuclear cell (PBMC) composition in the form of a solid, comprising

A) a cryoprotectant, which in illustrative embodiments is or comprises trehalose, wherein in certain embodiments the composition is 1-50% (w/v) trehalose;

B) a lyoprotectant for example 25-75% (w/v) polysucrose and/or albumin;

C) PBMC cell culture media components comprising amino acids, vitamins, and inorganic salts which may function as a buffer, and optionally a second buffer component; and

D) a population of between 1×10⁶ and 1×10¹⁰ freeze-dried peripheral blood mononuclear cells (PBMCs), wherein at least 1% of the PBMCs in said population, when rehydrated are viable. In illustrative embodiments, the composition when rehydrated to a target volume comprises the PBMC cell culture media components at concentrations that effectively support PBMC cell culturing. In further embodiments, the cell culture media components can be selected from those found in Roswell Park Memorial Institute medium (RPMI) 1640 medium, Iscove's modified Dulbecco's medium (IMDM), Dulbecco's modified Eagle medium (DMEM), McCoy's 5 A medium, minimum essential medium alpha medium (alpha-MEM), basal medium Eagle (BME), Fischer's medium, medium 199, and F-12K nutrient mixture medium (Kaighn's modification, F-12K). In further embodiments the cell culture medium can be a custom modification of RPMI 1640 available from e.g. ThermoFisher Scientific (see: www.thermofisher.com/us/en/home/life-science/cell-culture/mammalian-cell-culture/classical-media.html).

In one aspect, provided herein is a peripheral blood mononuclear cell (PBMC) composition in liquid form, comprising a population of between 1×10⁶ and 1×10¹⁰ freeze-dried peripheral blood mononuclear cells (PBMCs) in a PBMC cell culture media; trehalose at a concentration of from 0.1% (w/v) to 10.0% (w/v), polysucrose and/or albumin at a concentration from 0.1% (w/v) to 15% (w/v); and wherein between 1% and 25% of the PBMCs in said population are viable.

In one aspect, provided herein is a peripheral blood mononuclear cell (PBMC) composition in the form of a solid, comprising

A) trehalose, wherein when the composition is rehydrated, the trehalose is present at a concentration of from 0.1% (w/v) to 10.0% (w/v);
B) either one or both polysucrose and albumin, wherein when the composition is rehydrated the polysucrose and albumin are independently or in combination, present at a concentration from 0.1% (w/v) to 15% (w/v);
C) DMSO in a concentration of from 1% to 5%; and
D) a population of between 1×10⁶ and 1×10¹⁰ freeze-dried peripheral blood mononuclear cells (PBMCs), wherein when the composition is rehydrated, between 1% and 25% of the PBMCs in said population are viable.

In one aspect, provided herein is a peripheral blood mononuclear cell (PBMC) composition in liquid form, comprising:

A) trehalose, wherein trehalose is present at a concentration of from 0.1% (w/v) to 10.0% (w/v);

B) either one or both polysucrose and albumin, wherein when the polysucrose and albumin are independently or in combination, present at a concentration from 0.1% (w/v) to 15% (w/v);

C) DMSO in a concentration of from 1% to 5%; and

D) a population of between 1×10⁶ and 1×10¹⁰ peripheral blood mononuclear cells (PBMCs), wherein between 1% and 25% of the PBMCs in said population are viable.

In an illustrative embodiment, that is supported by the data provided in the Examples for Formulation 3, provided herein is a PBMC composition in the form of a solid comprises between 10-20% trehalose, between 50-70% polysucrose, 10-20% DMSO, and 0.05-0.2% and/or between 1×10⁵ and 1×10¹¹ dried, in illustrative embodiments freeze-dried PBMCs. In another illustrative embodiment, provided herein is a PBMC composition in the form of a solid comprises between 14-18% trehalose, between 60-70% polysucrose, 12-18% DMSO, and 0.05-0.15% and/or between 1×10⁶ and 1×10¹⁰ dried, in illustrative embodiments freeze-dried PBMCs. Such illustrative embodiments supported by Formula 3 can further include a buffer at an effective amount upon rehydration of the composition, inorganic sales, for example at between 1 and 2%, additional sugars, for example dextrose, at a concentration of 0.2 to 0.6%, and a serum source, such as fetal bovine serum, at a concentration of 0.02 to 1%, or 0.04 to 0.06%. Such illustrative embodiments supported by Formula 3 can further include any of the other components provided in Table 9 at concentrations +/−20, 15, 10, or 5% those provided in Formula 3. In further illustrative embodiments, between 15 and 25%, or 15 and 20% of the freeze-dried PBMCs in the composition are viable, for example as demonstrated when rehydrated.

In an illustrative embodiment that is supported by the data provided in the Examples for Formulation 5, provided herein is a PBMC composition in the form of a solid that includes between 40-50% trehalose, between 40-50% albumin (e.g. bovine serum albumin (BSA) or human serum albumin (HSA)), and dried, typically freeze-dried PBMCs, for example as 0.1-0.3% of the solid and/or between 1×10⁵ and 1×10¹¹ dried, in illustrative embodiments freeze-dried PBMCs. In another illustrative embodiment, provided herein is a PBMC composition in the form of a solid comprises between 42-48% trehalose, between 42-48% BSA, and dried, typically freeze-dried PBMCs, for example as 0.12-0.25% of the solid and/or between 1×10⁶ and 1×10¹⁰ dried, in illustrative embodiments freeze-dried PBMCs. Such illustrative embodiments supported by Formula 5 can further include a buffer at an effective amount upon rehydration of the composition, inorganic salts, for example, at between 8 and 12%, and a serum source, such as fetal bovine serum, at a concentration of 0.02 to 0.15%, or 0.04 to 0.12%. In some embodiments, the inorganic salts include sodium chloride as 6-10%, 7-9%, or about 8% of the solid. Such illustrative embodiments supported by Formula 5 can further include any of the other components provided in Table 11 at concentrations +/−20, 15, 10, 5, or 1% those provided in Formula 5. In further illustrative embodiments, between 0.5 and 5%, or 0.75 and 2% of the freeze-dried PBMCs in the compositions are viable, as demonstrated for example when rehydrated.

In another illustrative embodiment, that is supported by the data provided in the Examples for Formulation 6, provided herein is a PBMC composition in the form of a solid comprises between 10-20% trehalose, between 10-20% sorbitol, between 20 and 30% albumin, between 25 and 50% polysucrose, and 0.05-0.2% and/or between 1×10⁵ and 1×10¹¹ dried, in illustrative embodiments freeze-dried PBMCs. In another illustrative embodiment, that is supported by the data provided in the Examples for Formula 6, provided herein is a PBMC composition in the form of a solid comprises between 14-18% trehalose, between 12-18% sorbitol, between 25 and 30% albumin, between 25 and 35% polysucrose, and 0.05-0.15% and/or between 1×10⁵ and 1×10¹⁰ dried, in illustrative embodiments freeze-dried PBMCs. In illustrative embodiments, such compositions do not include any DMSO. In further embodiments of such illustrative embodiments supported by Formula 6, the freeze-dried PBMC composition further comprises PBMC cell culture media components comprising a buffer, amino acids, vitamins, and inorganic salts. Such PBMC cell culture media components can be present at concentrations that provide an effective cell culture media for culturing PBMCs when such solid freeze-dried PBMC composition is rehydrated in a target volume, which in illustrative embodiments is the volume of the composition at the start of the freeze-drying process. Such illustrative embodiments supported by Formula 6 can further include any of the other components provided in Table 12 at concentrations +/−20, 15, 10, or 5% those provided in Formula 6. In some embodiments the cell culture media components are dehydrated RPMI-1640 cell culture media. In further illustrative embodiments, between 5 and 10%, or 5 and 8% of the freeze-dried PBMCs in the composition are viable, as demonstrated for example when rehydrated.

Provided herein in some aspects, are methods for preparing freeze-dried nucleated cells (e.g. PBMCs). It will be understood, as demonstrated in the Examples section herein, for example using the MTT assay, that processes/methods for preparing freeze-dried nucleated cells (e.g. PBMCs), in certain embodiments, result in populations of freeze-dried nucleated cells (e.g. PBMCs) wherein at least some of the nucleated cells (e.g. PBMCs) in the population have preserved or even enhanced metabolic activity when such population of freeze-dried nucleated cells (e.g. PBMCs) are tested after rehydration. Thus, it will be understood that methods for preparing freeze-dried nucleated cells (e.g. PBMCs) herein, in some aspects are methods for preparing freeze-dried nucleated cells (e.g. PBMCs) with preserved or enhanced metabolic activity Similarly, it will be understood that methods of preparing freeze-dried nucleated cells, in illustrative embodiments PBMCs, provided herein prepare populations of freeze-dried nucleated cells (e.g. PBMCs) in which some of the freeze-dried cells in the population are viable. Thus, it will be understood that methods for preparing freeze-dried nucleated cells (e.g. PBMCs) herein, in some aspects are methods for preserving nucleated cells (e.g. PBMCs), or methods for preserving viability of freeze-dried nucleated cells (e.g. PBMCs).

In another aspect, provided herein is a method of preparing freeze-dried peripheral blood mononuclear cells PBMCs the method comprising:

A) incubating PBMCs, in some embodiments at a temperature between 20° C. and 40° C., 25° C. and 40° C., 27° C. and 42° C., 32° C. and 42° C., or 35° C. and 39° C., or at about or exactly 37° C., in certain embodiments for between 5 minutes and 8 hours, for example between 10 minutes and 2 hours, between 30 minutes and 2 hours, suspended in a mixture comprising polysucrose, trehalose, sorbitol, and albumin in an aqueous environment to form incubated PBMCs suspended in the mixture; and

B) lyophilizing the incubated PBMCs suspended in the mixture to form a solid composition comprising freeze-dried PBMCs. Such solid composition is typically a powder with less than 1% water content.

In another aspect, provided herein is a method of preparing freeze-dried peripheral blood mononuclear cells (PBMCs), the method comprising:

A. incubating PBMCs at a temperature of between 35° C. and 40° C. for between 10 minutes and 120 minutes, in an aqueous mixture to form incubated PBMCs suspended in the mixture, wherein the mixture comprises i) trehalose, wherein the trehalose is present in the aqueous mixture at a concentration of from 0.1% (w/v) to 10.0% (w/v); ii) an excipient comprising polysucrose and/or albumin, wherein the polysucrose and/or the albumin are present in the aqueous medium, at a concentration individually or in combination from 4.0% (w/v) to 20.0% (w/v); iii) a buffer; and either one or both iv) sorbitol and PBMC cell culture media components, wherein the sorbitol if present, is present in the aqueous medium at a concentration from 0.1% (w/v) to 5.0% (v/v), and the cell culture media components if present comprise amino acids, vitamins, and inorganic salts, at effective concentrations to facilitate culturing of PBMCs; and B. lyophilizing the incubated PBMCs suspended in the mixture to form a solid composition comprising freeze-dried PBMCs.

In one aspect of preparing, using or administering freeze-dried or rehydrated peripheral blood mononuclear cells (PBMCs) the aqueous mixture comprises a PBMC cell culture media and a buffer that comprises the buffer of the cell culture media.

In further aspects of preparing freeze-dried peripheral blood mononuclear cells (PBMCs), the method comprising: A. incubating between 1×10⁶ and 1×10¹² freeze-dried PBMCs at a temperature of between 35 C and 40 C for between 10 minutes and 120 minutes, in an aqueous medium comprising trehalose at a concentration of from 0.1% (w/v) to 10.0% (w/v), an excipient comprising polysucrose and/or albumin at a concentration from 0.1% (w/v) to 15% (w/v), and DMSO at a concentration of from 2% to 5%, to form incubated PBMCs suspended in the mixture; and B. lyophilizing the incubated PBMCs suspended in the mixture to form a solid composition comprising freeze-dried PBMCs.

In one embodiment of the method of preparing or administering freeze-dried peripheral blood mononuclear cells (PBMCs), the method further comprises rehydrating the solid composition comprising freeze-dried PBMCs to form a liquid composition comprising rehydrated PBMCs wherein between 1% and 20%, for example between 1 and 15%, between land 10%, or between 1 and 8% of the freeze-dried PBMCs are viable in the liquid composition. In some embodiments of such methods, the PBMCs are not in contact with DMSO during the method of preparing freeze-dried PBMCs and/or have not been exposed to DMSO during the method and/or are not suspended in a liquid comprising DMSO during the method. In some embodiments between 1% and 10% of the freeze-dried PBMCs are viable in the liquid composition comprising rehydrated freeze-dried PBMCs.

In some embodiments of PBMC compositions herein, DMSO is not present in the PBMC composition.

In one aspect, the liquid PBMC composition, or when the solid PBMC composition is rehydrated, between 1% and 10% of the freeze-dried cells are viable.

In some embodiments of a method of preparing freeze-dried PBMCs, wherein DMSO is present in the mixture in which PBMCs are lyophilized, the method further comprises rehydrating the solid composition comprising freeze-dried PBMCs to form a liquid composition comprising rehydrated platelets. Such methods can be referred to as methods for preparing freeze-dried PBMCs and rehydrating the freeze-dried PBMCs. In certain embodiments of such a method, between 2% and 25%, 5% and 25%, 10% and 25%, 15% and 25%, 5% and 20%, 10% and 20%, or 15% and 20% of the freeze-dried PBMCs are viable in the liquid composition comprising rehydrated platelets.

In one aspect, provided herein is a PBMC composition wherein when the solid PBMC composition is rehydrated and between 5% and 25% of the freeze-dried cells are viable.

In one aspect of the method, the solid is a powder, wherein the powder is a freeze-dried powder, and wherein the composition comprises less than 1% water content.

In some embodiments, the composition is present in a plurality of containers. Such composition can be a regulatory agency-approved product, such as a regulatory agency approved biologic.

In certain embodiments, a method herein can further include isolating PBMCs using a method that includes an anti-coagulant, for example acid citrate dextrose (ACD-A), but does not comprise heparin-treated blood. In certain embodiments, the dried or liquid composition, the mixture, or the aqueous medium does not comprise fibrinogen. In certain embodiments of any method herein, the method does not comprise heating the solid composition comprising freeze-dried PBMCs to a temperature above 50, 55, 56, 57, 58, or 59, in certain embodiments for more than 1, 2, 3, 4, 5, 10, 15, 20, 30 or 45 minutes, or for more than 1, 2, 3, 4, 5, 6, 7, or 8 hours. In certain embodiments, the methods herein do not comprise heating the solid composition comprising freeze-dried PBMCs to a temperature of about or exactly 60 C or above for more than 1 hour.

In one aspect of the method of preparing freeze-dried PBMCs, the incubating is performed for between 15 min and 60 min at about 37 C.

In one aspect, provided herein is a PBMC composition or a method of preparing, using, or administering freeze-dried PBMCs or rehydrated PBMCs, wherein the trehalose is present in the PBMC composition, the aqueous mixture, or the aqueous medium at a concentration of from 2.0% (w/v) to 6.0% (w/v).

In one aspect, provided herein is a PBMC composition or a method of preparing, using or administering freeze-dried PBMCs or rehydrated PBMCs, wherein the PBMC composition, the aqueous mixture, or the aqueous medium, includes sorbitol at a concentration from about or exactly 0.1% (w/v) to about or exactly 5.0% (w/v).

In one aspect, provided herein is a PBMC composition or a method of preparing, using or administering freeze-dried PBMCsor rehydrated PBMCs, wherein the PBMC composition, the aqueous mixture, or the aqueous medium, includes albumin at a concentration from 3.0% (w/v) to 7.0% (w/v).

In one aspect, provided herein is a PBMC composition or a method of preparing, using or administering freeze-dried PBMCsor rehydrated PBMCs, wherein the PBMC composition, the aqueous mixture, or the aqueous medium, includes polysucrose at a concentration from 3.0% (w/v) to 7.0% (w/v).

In one aspect, provided herein is a PBMC composition or a method of preparing, using or administering freeze-dried PBMCsor rehydrated PBMCs, wherein the PBMC composition, the aqueous mixture, or the aqueous medium, includes trehalose at a concentration of from 2.0% (w/v) to 6.0% (w/v) and polysucrose is present at a concentration from 3.0% (w/v) to 7.0% (w/v).

In one aspect, provided herein is a PBMC composition wherein the lyophilized PBMCs have metabolic activity.

In one aspect, provided herein, the lyophilized PBMCs have enhanced metabolic activity compared to lyophilized PBMCs lyophilized in a medium comprising 2% to 3% DMSO.

In one aspect, provided herein is a PBMC composition wherein sorbitol and albumin are present in the PBMC composition, the aqueous mixture, or the aqueous medium, wherein sorbitol is present at a concentration from 1% (w/v) to 5.0% (w/v) and wherein albumin is present at a concentration from 3.0% (w/v) to 7.0% (w/v).

In one aspect, provided herein are lyophilized PBMCs that have enhanced metabolic activity compared to lyophilized PBMCs lyophilized in a medium comprising 2% to 3% DMSO.

In one aspect, provided herein is a PBMC composition or a method of preparing, using, or administering freeze-dried PBMCs, wherein the PBMC composition, the aqueous mixture, or the aqueous medium comprises polysucrose at a concentration from 10% (w/v) to 15% (w/v).

In embodiments of any of the methods/processes and compositions provided herein, the aqueous mixture and/or the rehydrated platelet composition comprises a PBMC cell culture media, or components thereof. In some embodiments, when dried compositions provided herein, are rehydrated to a target volume, the resulting liquid composition comprises the PBMC cell culture media components at concentrations that effectively support PBMC cell culturing, and illustrative embodiments are at their target concentrations for that particular media. In further embodiments, the cell culture media components can be selected from those found in Roswell Park Memorial Institute medium (RPMI) 1640 medium, Iscove's modified Dulbecco's medium (IMDM), Dulbecco's modified Eagle medium (DMEM), McCoy's 5 A medium, minimum essential medium alpha medium (alpha-MEM), basal medium Eagle (BME), Fischer's medium, medium 199, and F-12K nutrient mixture medium (Kaighn's modification, F-12K). In certain illustrative embodiments, the cell culture media is RPMI-1640 cell culture media. In yet further embodiments the cell culture medium can be a custom modification of RPMI 1640 available from e.g. ThermoFisher Scientific (see: www.thermofisher.com/us/en/home/life-science/cell-culture/mammalian-cell-culture/classical-media/rpmi.html).

In one aspect of a method, for example methods of using or administering freeze-dried PBMC produced by any of these methods, the method further comprises rehydrating the lyophilized PBMCs.

In one aspect of a method, trehalose is present in the aqueous mixture at a concentration of from 2.0% (w/v) to 6.0% (w/v) and polysucrose is present at a concentration from 3.0% (w/v) to 7.0% (w/v).

In one aspect of a method, sorbitol and albumin are present in the aqueous mixture, wherein sorbitol is present at a concentration from 1% (w/v) to 5.0% (w/v) and wherein albumin is present at a concentration from 3.0% (w/v) to 7.0% (w/v).

In one aspect of a method, the lyophilized PBMCs have enhanced metabolic activity compared to lyophilized PBMCs lyophilized in a medium comprising 2% to 3% DMSO.

Provided herein are populations of freeze-dried peripheral mononuclear blood cells (PBMCs), where said population, when rehydrated has a viability level of at least 1%.

In some embodiments, the population has a viability level of at least 3%. In some embodiments, the population has a viability level of at least 5%.

In some embodiments, the PBMCs are mammalian cells. In some embodiments, the PBMCs are human cells. In some embodiments, the PBMCs are lymphocytes, monocytes, or granulocytes. In some embodiments, the lymphocytes are B-cells or T-cells.

Also provided herein are methods of preparing freeze-dried PBMCs, the method including: incubating PBMCs in a buffered aqueous environment that includes at least a buffer, a salt, and a sugar, and optionally a bulking agent to form a mixture; and lyophilizing the mixture to form freeze-dried PBMCs.

Also provided herein are methods of preparing freeze-dried PBMCs, the method including: incubating PBMCs with a mixture including polysucrose, trehalose, sorbitol, and BSA in an aqueous environment to form incubated PBMCs; and lyophilizing the mixture to form freeze-dried PBMCs.

In some embodiments, the incubating buffer includes a cell culture media that is effective for culturing PBMCs. In certain illustrative embodiments, the media is RPMI-1640 cell culture media. In further embodiments, the cell culture media components can be selected from those found in Roswell Park Memorial Institute medium (RPMI) 1640 medium, Iscove's modified Dulbecco's medium (IMDM), Dulbecco's modified Eagle medium (DMEM), McCoy's 5 A medium, minimum essential medium alpha medium (alpha-MEM), basal medium Eagle (BME), Fischer's medium, medium 199, and F-12K nutrient mixture medium (Kaighn's modification, F-12K). In further embodiments the cell culture medium can be a custom modification of RPMI 1640 available from e.g. ThermoFisher Scientific (see: www.thermofisher.com/us/en/home/life-science/cell-culture/mammalian-cell-culture/classical-media/rpmi.html).

In some embodiments herein that include a composition, or in some compositions used in or formed by a process that includes PBMCs (e.g. a population thereof) in a hydrated or rehydrated form, such composition comprises trehalose in the range of 0.4-35%, 1-35%, 2-30%, 0.1-10%, 1-10%, 1-5%, 1-4%, 2-6%, 2-5%, 2-4%, or 0.5-5%. In an exemplary embodiment, the composition comprises 3.5% trehalose.

In some embodiments herein that include a composition, or in some compositions used in or formed by a process that includes a PBMC composition in a powdered form, the composition comprises trehalose having a weight percentage in the range of 1-60%, 1-50%, 1-40%, 10-50%, 10-60%, 15-55%, 20-60%, 20-50%, 25-60%, 25-50%, 10-50%, 20-40%, or 20-35%. In some embodiments, the weight percentage of trehalose can vary on the weight percentage of other components in the composition like, polysucrose, PBMCs, cell culture media components, sorbitol and/or albumin.

In some embodiments herein that include a composition, or in some compositions used in or formed by a process that includes PBMCs (e.g. a population thereof) in a hydrated or rehydrated form, the composition comprises polysucrose in the range of 2-20%, 2-15%, 3-15%, 4-15%, 5-15%, or 6-14%, 2-14%, 3-14%, 4-14%, 5-14%, or 6-14%, 6-13%, 2-13%, 3-13%, 4-13%, 5-13%, or 6-13%, 2-7%, 3-7%, 4-7%, 2.5-7.5%, or 2.5-6.5%. In an exemplary embodiment, the composition comprises 3% polysucrose. In another exemplary embodiment, the composition comprises 6% polysucrose.

In some embodiments herein that include a composition, or in some compositions used in or formed by a process that includes a PBMC composition in a powdered form, comprises polysucrose having a weight percentage in the range of 20-80%, 25-75%, 30-70%, 35-65%, 30-80%, or 45-60%. In some embodiments, the weight percentage of trehalose can vary on the weight percentage of other components in the composition like, trehalose, PBMCs, plasma protein, and buffering agents.

In some embodiments herein that include a composition, or in some compositions used in or formed by a process that includes a PBMC composition in a solid (e.g. powdered) form, the composition comprises trehalose and polysucrose having a combined weight percentage in the range of 10-90%, 10-80%, 10-75%, 10-70%, 10-60%, 10-50%, 30-95%, 35-95%, 40-90%, 40-90%, 45-90%, or 60-95%. A skilled artisan will understand that the percent of all the components of the composition in solid form cannot exceed 100%.

In some embodiments herein that include a composition or in some compositions used in or formed by a process herein, the composition comprises polysucrose, the polysucrose is a cationic form of polysucrose. In some embodiments, the cationic form of polysucrose is diethylaminoethyl (DEAE)-polysucrose. In some embodiments, the polysucrose is an anionic form of polysucrose. In some embodiments, the anionic form of polysucrose is carboxymethyl-polysucrose. In some embodiments of the composition, polysucrose has a molecular weight in the range of 70,000 MW to 400,000 MW, 100,000 MW to 400,000 MW, 200,00 MW to 400,000 MW, 80,000 MW to 350,000 MW, 100,000 MW to 300,00 MW, 100,000 MW to 200,000 MW, 120,000 MW to 200,000 MW. In some exemplary embodiments, polysucrose has a molecular weight of 150,000 MW, 160,000 MW, 170,000 MW, 180,000 MW, 190,000 MW, or 200,000 MW.

In some embodiments, albumin (e.g., BSA or HSA) is present from about or exactly 0.01% (w/v) to about or exactly 10.0% (w/v), from about or exactly 0.1% (w/v) to about or exactly 7.0% (w/v), from about or exactly 0.5% (w/v) to about or exactly 6.5% (w/v), from about or exactly 1.0% (w/v) to about or exactly 6.0% (w/v), from about or exactly 1.5% (w/v) to about or exactly 5.5% (w/v), from about or exactly 2.0% (w/v) to about or exactly 5.0% (w/v), from about or exactly 2.5% (w/v) to about or exactly 4.5% (w/v), from about or exactly 3.0% (w/v) to about or exactly 4.0% (w/v), or about or exactly 3.5% (w/v).

In some embodiments herein that include a composition or in some compositions used in or formed by a process herein, the trehalose is present in the aqueous environment at a concentration of from about or exactly 0.1% (w/v) to about or exactly 10.0% (w/v). In some embodiments, the trehalose is present in the aqueous environment at a concentration of from about or exactly 1.0% (w/v) to about or exactly 5.0% (w/v). In some embodiments, the sorbitol is present in the aqueous environment at a concentration from about or exactly 0.1% (w/v) to about or exactly 5.0% (w/v). In some embodiments, the BSA is present in the aqueous environment at a concentration from about or exactly 1.0% (w/v) to about or exactly 10.0% (w/v). In some embodiments, the BSA is present in the aqueous environment at a concentration from about or exactly 3.0% (w/v) to about or exactly 7.0% (w/v). In some embodiments, the polysucrose is present in the aqueous environment at a concentration from about or exactly 0.1% (w/v) to about or exactly 15% (w/v). In some embodiments, the polysucrose is present in the aqueous environment at a concentration from about or exactly 3.0% (w/v) to about or exactly 7.0% (w/v).

Also provided herein are rehydrated freeze-dried PBMCs produced by any of the methods described herein, where the method includes rehydrating the lyophilized PBMCs.

Also provided herein are freeze-dried PBMCs prepared by a process including the steps of: incubating PBMCs in an aqueous environment that includes at least a buffer, a salt, and a sugar, and optionally a bulking agent to form a mixture; and lyophilizing the mixture to form freeze-dried PBMCs.

Also provided herein are freeze-dried PBMCs prepared by a process including the steps of: incubating PBMCs with a mixture including polysucrose, trehalose, sorbitol, and BSA in an aqueous environment to form incubated PBMCs; and lyophilizing the mixture, to form freeze-dried PBMCs.

Also provided herein are freeze-dried PBMCs prepared by any of the processes described herein the trehalose is present in the aqueous environment at a concentration of from about or exactly 0.1% (w/v) to about or exactly 10.0% (w/v); the sorbitol is present in the aqueous environment at a concentration from about or exactly 0.1% (w/v) to about or exactly 5.0% (w/v); the BSA is present in the aqueous environment at a concentration from about or exactly 0.1% (w/v) to about or exactly 10.0% (w/v); and the polysucrose is present in the aqueous solution at a concentration from about or exactly 0.1% (w/v) to about or exactly 15% (w/v).

Also provided herein are freeze-dried PBMCs prepared by any of the processes described herein, where the trehalose is present in the aqueous environment at a concentration of from about or exactly 1.0% (w/v) to about or exactly 5.0% (w/v); the sorbitol is present in the aqueous environment at a concentration of from about or exactly 2.5% (w/v) to about or exactly 3.5% (w/v); the BSA is present in the aqueous environment at a concentration from about or exactly 3.0% (w/v) to about or exactly 7.0% (w/v); and the polysucrose is present in the aqueous solution at a concentration from about or exactly 3.0% (w/v) to about or exactly 7.0% (w/v).

Also provided herein are freeze-dried PBMCs prepared by any of the processes described herein, where the process includes admixing RPMI-1640.

Also provided herein freeze-dried PBMCs prepared by any of the processes described herein, where the population has a viability level of at least 1%. Also provided herein are freeze-dried PBMCs prepared by any one of the processes described herein, where the population has a viability level of at least 3%. Also provided herein are freeze-dried PBMCs prepared by any one of the processes described herein, where the population has a viability level of at least 5%.

Also provided herein are methods of preserving viability of a population of PBMCs including: contacting PBMCs with a mixture including trehalose, sorbitol, BSA, and polysucrose in an aqueous environment to form a population of incubated PBMCs with preserved viability; and lyophilizing the mixture to form a population of incubated PBMCs with preserved viability.

Also provided herein are methods of preserving viability of a population of PBMCs, the method including: incubating the population of PBMCs with a mixture including, trehalose, sorbitol, BSA, and optionally a bulking agent in an aqueous environment to form incubated PBMCs; and lyophilizing the mixture to form a population of incubated PBMCs with preserved viability.

In another aspect, provided herein is a method of enhancing % max activity, or a method of enhancing metabolic activity of a population of PBMCs, the method comprising: incubating the population of PBMCs with a mixture comprising trehalose, sorbitol, BSA, and optionally a bulking agent in an aqueous environment to form a population of PBMCs with enhanced % max activity, or with enhanced metabolic activity, respectively.

Thus, in some embodiments, provided herein is a method of enhancing % maximum (“max”) activity, or a method of enhancing metabolic activity of a population of PBMCs, the method comprising:

contacting PBMCs with a mixture comprising trehalose, sorbitol, BSA, and polysucrose in an aqueous environment to form a population of PBMCs with enhanced % max activity, or with enhanced metabolic activity, respectively.

For example, methods of enhancing % max activity, or a method of enhancing metabolic activity of a population of PBMCs (e.g., fresh PBMCs) include incubating the population of fresh PBMCs in a solution (e.g., buffer) derived from rehydrated PBMCs. In some embodiments, PBMCs lyophilized in any of the lyophilization formulations described herein (e.g., formulations 1-6) are rehydrated. In some embodiments, PBMCs lyophilized in formulation 6 are rehydrated. In some embodiments, the rehydrated PBMCs are removed by centrifugation and/or filtration. In such embodiments, the resulting solution after centrifugation and/or filtration, when incubated with a population of fresh PBMCs, results in enhanced % max activity of the population of PBMCs (e.g., fresh PBMCs).

Thus, in some embodiments, provided herein is a method of preserving % max activity or a method of preserving metabolic activity of a population of PBMCs, the method comprising: incubating the population of PBMCs with a first mixture comprising trehalose, sorbitol, and BSA in an aqueous environment to form incubated PBMCs; and contacting the incubated PBMCs with polysucrose to form a population of PBMCs with preserved % max activity or metabolic activity, respectively.

Thus, provided herein, is a method of enhancing % max activity or a method of enhancing metabolic activity of a population of PBMCs, the method comprising: contacting PBMCs with a mixture comprising trehalose, sorbitol, BSA, and polysucrose in an aqueous environment to form a population of PBMCs with enhanced % max activity or enhanced metabolic activity, respectively.

Thus, provided herein, is a method of enhancing % max activity or a method of enhancing metabolic activity of a population of PBMCs, the method comprising: incubating the population of PBMCs with a mixture comprising polysucrose, trehalose, and DMSO in an aqueous environment to form a population of PBMCs with enhanced % max activity, or enhanced metabolic activity, respectively.

Thus, provided herein, is a method of enhancing % max activity of a population of PBMCs or a method of enhancing metabolic activity, the method comprising: contacting PBMCs with a mixture comprising trehalose and BSA in an aqueous environment to form a population of PBMCs with enhanced % max activity, or enhanced metabolic activity, respectively.

Thus, provided herein, is a method of enhancing % max activity, or a method of enhancing metabolic activity of a population of PBMCs, the method comprising: incubating the population of PBMCs with a mixture comprising trehalose and BSA in an aqueous environment to form a population of PBMCs with enhanced % max activity, or enhanced metabolic activity, respectively.

The components of the mixtures and incubation conditions for these methods related to % max activity or enhancing metabolic activity, are those provided herein, including, but not limited to those hereinabove in this section.

In some embodiments, the trehalose is present in the aqueous environment at a concentration of from about or exactly 0.1% (w/v) to about or exactly 10.0% (w/v). In some embodiments, the trehalose is present in the aqueous environment at a concentration of from about or exactly 1.0% (w/v) to about or exactly 8.0% (w/v). In some embodiments, the trehalose is present in the aqueous environment at a concentration of from about or exactly 3.0% (w/v) to about or exactly 6.0% (w/v). In some embodiments, the sorbitol is present in the aqueous environment at a concentration of from about or exactly 0.01% (w/v) to about or exactly 7.5% (w/v). In some embodiments, the sorbitol is present in the aqueous environment at a concentration from about or exactly 0.1% (w/v) to about or exactly 5.0% (w/v). In some embodiments, the sorbitol is present in the aqueous environment at a concentration from about or exactly 2.5% (w/v) to about or exactly 3.5% (w/v). In some embodiments, the BSA is present in the aqueous environment at a concentration from about or exactly 0.1% (w/v) to about or exactly 10.0% (w/v). In some embodiments, the BSA is present in the aqueous environment at a concentration from about or exactly 3.0% (w/v) to about or exactly 7.0% (w/v). In some embodiments, the polysucrose is present in the aqueous solution at a concentration from about or exactly 0.1% (w/v) to about or exactly 15% (w/v). In some embodiments, the polysucrose is present in the aqueous solution at a concentration from about or exactly 3.0% (w/v) to about or exactly 7.0% (w/v). In some embodiments, the incubating buffer includes RPMI-1640 cell culture media.

In some embodiments, the method includes lyophilizing the population of PBMCs.

In some embodiments, the population has a % max activity of at least about 15%. In some embodiments, the population has a % max activity of at least about 20%. In some embodiments, the population has a % max activity of at least about 25%. In some embodiments, the population has a % max activity of at least about 30%. In some embodiments, the population has a % max activity of at least about 35%. In some embodiments, the population has a % max activity of at least about 40%.

In some embodiments, the PBMCs are mammalian PBMCs. In some embodiments, the PBMCs are human PBMCs. In some embodiments, the PBMCs are lymphocytes or monocytes. In some embodiments, the lymphocytes are B-cells or T-cells.

Also provided herein are methods of preparing freeze-dried PBMCs, the method including: incubating PBMCs with a mixture including 6% (w/v) polysucrose, 3% (w/v) trehalose, 3% (w/v) sorbitol, and 5% (w/v) BSA in an aqueous environment to form incubated PBMCs; and lyophilizing the mixture to form freeze-dried PBMCs.

Also provided herein are freeze-dried PBMCs prepared by a process including the steps of: incubating PBMCs with a mixture including 6% (w/v) polysucrose, 3% (w/v) trehalose, 3% (w/v) sorbitol, and 5% (w/v) BSA in an aqueous environment to form incubated PBMCs; and lyophilizing the mixture, to form freeze-dried PBMCs.

Also provided herein are methods of preparing freeze-dried PBMCs, the method including: incubating PBMCs with a mixture including polysucrose, trehalose, and DMSO in an aqueous environment to form incubated PBMCs; and lyophilizing the mixture to form freeze-dried PBMCs.

In some embodiments, the polysucrose is present in the aqueous environment at a concentration from about or exactly 10% (w/v) to about or exactly 20% (w/v). In some embodiments, the DMSO is present in aqueous the environment at a concentration from about or exactly 1% (v/v) to about or exactly 5% (v/v).

Also provided herein are methods of preparing freeze-dried PBMCs, the method including: incubating PBMCs with a mixture including 13% (w/v) polysucrose, 3% (w/v) trehalose, and 2.5% (v/v) DMSO in an aqueous environment to form incubated PBMCs; and lyophilizing the mixture to form freeze-dried PBMCs.

Also provided herein are rehydrated freeze-dried PBMCs produced by any of the methods described herein, where the method includes rehydrating the lyophilized PBMCs.

Also provided herein are freeze-dried PBMCs prepared by a process including the steps of: incubating PBMCs with a mixture including polysucrose, trehalose, and DMSO in an aqueous environment to form incubated PBMCs; and lyophilizing the mixture, to form freeze-dried PBMCs.

Also provided herein are methods of preserving viability of a population of PBMCs, the method including: contacting PBMCs with a mixture including polysucrose, trehalose, and DMSO in an aqueous environment to form a population of incubated PBMCs with preserved viability; and lyophilizing the mixture to form a population of incubated PBMCs with preserved viability.

Also provided herein are methods of preserving viability of a population of PBMCs, the method including: incubating the population of PBMCs with a mixture including polysucrose, trehalose, and DMSO in an aqueous environment to form incubated PBMCs; and lyophilizing the mixture to form a population of incubated PBMCs with preserved viability.

Also provided herein are rehydrated freeze-dried PBMCs produced by any of the methods described herein, where the method includes rehydrating the lyophilized PBMCs.

Also provided herein are freeze-dried PBMCs prepared by a process including the steps of: incubating PBMCs with a mixture including trehalose and BSA in an aqueous environment to form incubated PBMCs; and lyophilizing the mixture, to form freeze-dried PBMCs.

Also provided herein are methods of preserving viability of a population of PBMCs, the method including: contacting PBMCs with a mixture including trehalose and BSA in an aqueous environment to form a population of incubated PBMCs with preserved viability; and lyophilizing the mixture to form a population of incubated PBMCs with preserved viability.

Also provided herein are method of preserving viability of a population of PBMCs, the method including: incubating the population of PBMCs with a mixture including trehalose and BSA in an aqueous environment to form incubated PBMCs; and lyophilizing the mixture to form a population of incubated PBMCs with preserved viability.

It will be understood that any composition and method herein that includes and recites PBMCs, can instead include or recite any one or more of the cell-type components of PBMCs (e.g. T cells, NK cells, B cells, or monocytes) and genetically modified versions of PBMCs or any of these cell types.

Also provided herein in one aspect is a method of administering a rehydrated PBMC composition to a subject, the method comprising:

A) rehydrating a PBMC composition according to any freeze-dried PBMC embodiment herein to form the rehydrated PBMC composition; and

B) administering the rehydrated PBMC composition to the subject.

Also provided herein in one aspect is a biologic agent comprising rehydrated PBMCs for use in a method for administering a rehydrated PBMC composition to a subject, wherein the method comprises:

A) rehydrating a PBMC composition according to any freeze-dried PBMC embodiment herein to form the rehydrated PBMC composition; and

B) administering the rehydrated PBMC composition to the subject.

In such methods of administering and biologic agent use aspects, an effective amount of rehydrated PBMCs are delivered to the subject. Such administration is typically to treat a disorder or disorder that is treated by the rehydrated PBMCs.

The following non-limiting examples are provided purely by way of illustration of exemplary embodiments, and in no way limit the scope and spirit of the present disclosure. Furthermore, it is to be understood that any inventions disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

### EXAMPLES

### Example 1. Cell Surface Marker Presence and/or Expression on Fresh and Lyophilized PBMCs

The following formulations were tested to compare cell surface marker presence and/or expression between fresh PBMCs and lyophilized PBMCs:

A: Formulation A—formulation for cell stabilization containing HEPES-based buffer that includes trehalose, ethanol, and polysucrose (see Table 1).

TABLE 1
Formulation A

| | Concentration |
|---|---|
| Component | (mM unless otherwise specified) |
| NaCl | 60 |
| KCl | 3.84 |
| HEPES | 7.6 |
| NaHCO₃ | 9.6 |
| Dextrose | 2.4 |
| Trehalose | 80 |
| Ethanol | 0.8% (v/v) |
| Polysucrose | 6.0% (w/v) |

A: Formulation A alone

B: Formulation A, without ethanol

C: Formulation A, without ethanol with 2% Fibrinogen

D: Formulation A with 2% Fibrinogen

E: HBS (HEPES Buffered Saline) with 8% sucrose (negative control), includes 150 mM NaCl, 10 mM HEPES, adjusted to pH 7.4 with NaOH, and does not include trehalose, ethanol, or polysucrose.

The fresh PBMCs (see Example 2 for the source and preparation of PBMCs) were incubated separately in each of the described formulations above (formulations A-E) and subsequently lyophilized and rehydrated.

Loading, Lyophilization and Rehydration Method of PBMCs

The fresh PBMCs were diluted in the various Formulations (A-E) to 16.7 million cells per 1 mL (12 times dilution) and incubate at 37° C. for 30 minutes, with slight agitation. After the 37° C. incubation each Formulation was further diluted as specified below:

Sublots A, B, and E: Add 25% (volume equivalent) of appropriate buffer (cells were diluted to 12.5 million cells/mL)

Sublots C and D: Add 25% (volume equivalent) of 10% fibrinogen in appropriate formulation buffer (diluted to 12.5 million cells/mL)

Sublots A-D: add 20% (volume equivalent) of 30% polysucrose to each sublot (diluted to 10 million/mL cells).

Sublot E: add 20% solution of HBS/sucrose (10 million/mL cells).

A 1 mL sample of each PBMC formulation was placed in a vial and loaded into a lyophilizer on a prechilled shelf at −40° C. and the sample was lyophilized according to the protocol as described in Table A. A 1 mL vial of lyophilized PBMCs was rehydrated using 1 mL of cell culture grade water. The vial was incubated at room temperature for a total of 10 minutes. During the 10-minute rehydration period, the vial was gently swirled at 0, 5, and 10 minutes to promote dissolution of the lyophilizate.

Cell Surface Marker Assay

The PBMCs were analyzed for the presence of cell surface markers (CD19, CD45, CD3, CD14, CD4, CD8, CD56) by detection of fluorescent antibody binding using flow cytometry. Antibodies of interest were titrated using fresh PBMCs, and ideal concentrations were determined. Lyophilized samples of PBMCs were rehydrated and tested under similar conditions to determine if the surface receptors in question are retained.

Cell surface marker specific antibodies used were: Anti-CD19—Alexa Fluor 700 (Invitrogen Ref#56-0199-42); Anti-CD45-FITC (Invitrogen Ref#11-0459-42)); Anti-CD3-FITC (Invitrogen Ref# 11-0038-42); Anti-CD14-FITC (Invitrogen Ref#11-0149-42); Anti-CD4-Alexa Fluor 405 (Invitrogen Ref# MHCD0426); Anti-CD8-Alexa Fluor 700 (Invitrogen Ref# MIEICD0829); Anti-CD56-FITC (Invitrogen Ref# MA1-19574).

The fresh or rehydrated samples of PBMCs were incubated, i.e., stained, with the antibody, away from open light, at 4° C. for 30 minutes. After incubation 500 μL of cold PBS was added to each sample, 100 μL of each sample was transferred to a well in a 96 well U-bottom plate and data was captured using a Quanteon flow cytometer (NovoCyte Quanteon Flow Cytometer, Agilent Technologies, Santa Clara Calif., US).

The results of the percent positive cell surface marker presence and/or expression between fresh PBMCs and lyophilized PBMCs under different control buffer formulations are summarized in Table 2 below. The data was collected using flow cytometry and antigen specific antibodies.

TABLE 2
Percent Positive Summary

| Antigen | Fresh BMC | A | B | C | D | E |
|---|---|---|---|---|---|---|
| CD19 | 13.25% | 5.35% | 6.69% | 6.29% | 5.41% | 11.50% |
| CD45 | 70.76% | 61.19% | 64.82% | 55.83% | 58.15% | 49.11% |
| CD3 | 64.25% | 63.40% | 63.26% | 61.14% | 60.18% | 47.68% |
| CD14 | 20.40% | 15.35% | 14.34% | 12.61% | 10.67% | 10.97% |
| CD4 | 46.73% | 61.27% | 63.57% | 59.69% | 58.20% | 46.87% |
| CD8 | 26.76% | 24.52% | 25.06% | 12.70% | 12.28% | 15.51% |
| CD56 | 21.34% | 18.48% | 13.62% | 18.48% | 15.77% | 5.27% |

TABLE 3
MFI Summary

| Antigen | Fresh PBMC | A | B | C | D | E |
|---|---|---|---|---|---|---|
| CD19 | 6987 | 17719 | 15492 | 16273 | 18183 | 12322 |
| CD45 | 428062 | 201947 | 221971 | 182167 | 201550 | 168460 |
| CD3 | 93203 | 85271 | 92365 | 70958 | 81150 | 62126 |
| CD14 | 82261 | 61515 | 61097 | 53997 | 59656 | 45305 |
| CD4 | 74030 | 104028 | 88055 | 72130 | 68808 | 89849 |
| CD8 | 28825 | 25448 | 24499 | 42371 | 43005 | 24283 |
| CD56 | 16919 | 9514 | 12330 | 5212 | 5869 | 24434 |

The data in Table 2 represents the number of cells, tabulated as percent of lyophilized or fresh cells, expressing the cell surface antigen (e.g., CD19, CD45, CD3, CD14, CD4, CD8, CD56). The data in Table 3, mean fluorescence intensity (MFI), is a proxy for the number of antigen receptors. The results in Table 2 and Table 3 show that formulation A preserved more than 75% of surface marker positivity and more than 50% total MFI signal, respectively, relative to the fresh PBMC results (column A in both Table 2 and Table 3) for each cell surface marker (antigen) tested, except for CD19. Table 2 shows that about 40% of the CD19 cell surface marker were preserved with formulation A. Thus, overall, good preservation of cell surface markers after lyophilization and rehydration was achieved with formulation A. Formulation B, identical to formulation A but without ethanol, provided similar results to formulation A. Formulations C and D, which included 2% fibrinogen, appeared to provide lower percent positivity for a number of the markers (Table 2). Indeed, it was observed that fibrinogen caused cellular aggregation upon rehydration after lyophilization.

Without loading cells with cryopreservatives and incubating with other additives before lyophilization (negative control, column E), most cell surface marker positivity and MFI data are significantly reduced upon rehydration compared to the control formulation (formulation A), indicating a lack of cell preservation. Thus, the components of formulation A appear to be important to preserve cell function in lyophilized nucleated cells. The preservation of cell surface markers after rehydration of lyophilized cells that utilized the formulations with cryopreservatives tested herein (formulations A-D), especially formulations A and B, suggests that the lyophilized PBMCs will have function and be capable of responding to biochemical signaling similar to fresh PBMCs.

The results established that the formulations were effective for retaining cell surface markers after lyophilization and rehydration.

### Example 2. PBMC Stabilization Assays

Formulations 1-6 listed below were selected to identify formulations for lyophilization, and components thereof, that increased the effectiveness of the lyophilization process in terms of cell viability and activity of rehydrated nucleated cells. Accordingly, the following formulations were tested for lyophilized PBMC preservation: Formulation 1: Formulation A′—with 6% (w/v) Polysucrose (Table 1 control buffer) (positive control)

Formulation 2: Formulation A′—with 0.2 mg/mL EGCG (anti-oxidant) with 6% (w/v) Polysucrose

Formulation 3: Formulation A′—with 2.5% (v/v) DMSO and 13% (w/v) Polysucrose Formulation 4: Phosphate Buffer Saline (PBS) with 5% (w/v) Trehalose and 5% (w/v) Dextrose

Formulation 5: PBS with 5% (w/v) Trehalose and 5% (w/v) BSA Formulation 6: RPMI-1640 with 3% (w/v) Trehalose, 3% (w/v) Sorbitol, 5% (w/v) BSA and 6% (w/v) Polysucrose

As used above, “Formulation A′” refers to the Formulation A of Table 1 but without Polysucrose. Fresh human PBMCs (PBMNC300F) were supplied by Stem Express at a concentration of 200 million PBMCs/mL in PBS with 5% fetal bovine serum (FBS), 0.5% bovine serum albumin (BSA), and acid citrate dextrose (ACD-A) as the anticoagulant (see www.stemexpress.com/human-blood-products/human-peripheral-blood/peripheral-blood-leukopaks).

The fresh PBMCs were incubated separately in each of the described formulations above (formulations 1-6) and subsequently lyophilized and rehydrated.

Loading, Lyophilization and Rehydration Method of PBMCs

The fresh PBMCs were diluted in the in the various Formulations (1-6) to 5 million cells per 1 mL (40 times dilution) and incubate at 37° C. for 30 minutes, with slight agitation. A 1 mL sample of each PBMC formulation was placed in a vial and loaded into a lyophilizer on a prechilled shelf at −40° C. and the sample was lyophilized according to the protocol as described in Table A. A 1 mL vial of lyophilized PBMCs was rehydrated using 1 mL of cell culture grade water. The vial was incubated at room temperature for a total of 10 minutes. During the 10-minute rehydration period, the vial was gently swirled at 0, 5, and 10 minutes to promote dissolution of the lyophilizate.

Fresh PBMCs and lyophilized PBMCs preserved with the formulations described above were tested in a Trypan Blue Staining Assay. Cell viability was determined using a Trypan Blue exclusion test according to Strober, W. Trypan Blue Exclusion Test of Cell Viability, Current Protocols in Immunology, 1997, A.3B. 1-A.3B.2, which is herein incorporated by reference in its entirety.

The results of the Trypan Blue Staining Assays are summarized below for fresh PBMCs and lyophilized PBMCs in Table 4 and Table 5, respectively.

TABLE 4
Fresh PBMC Trypan Blue Staining Results

| Sample | Field of View | Clear | Blue | Total | Percent Viable |
|---|---|---|---|---|---|
| 1 | 1 | 79 | 2 | 81 | 97.5% |
| | 2 | 76 | 3 | 79 | 96.2% |
| | 3 | 84 | 2 | 86 | 97.7% |
| | 4 | 90 | 1 | 91 | 98.9% |
| | 5 | 71 | 4 | 75 | 94.7% |
| | 6 | 101 | 2 | 103 | 98.1% |
| 2 | 1 | 83 | 2 | 85 | 97.6% |
| | 2 | 85 | 1 | 86 | 98.8% |
| | 3 | 88 | 1 | 89 | 98.9% |
| | 4 | 75 | 3 | 78 | 96.2% |
| | 5 | 63 | 2 | 65 | 96.9% |
| | 6 | 66 | 4 | 70 | 94.3% |

| | | | | |
|---|---|---|---|---|
| All | 961 | 27 | 988 | 97.3% |

TABLE 5
Lyophilized PBMC Trypan Blue Staining Results

| | Number of | Number of | Total | Percent |
|---|---|---|---|---|
| Formulation | Clear Cells | Blue Cells | Cells | Viable |
| 1 | 0 | 123 | 123 | 0.0% |
| 2 | 0 | 95 | 95 | 0.0% |
| 3 | 30 | 121 | 151 | 19.9% |
| 4 | 0 | 123 | 123 | 0.0% |
| 5 | 3 | 310 | 313 | 1.0% |
| 6 | 17 | 237 | 254 | 6.7% |

The results in Table 4 demonstrate that fresh PBMCs are capable of excluding the Trypan blue dye, indicative of cell viability. For example, the lowest percent viable measurement observed was about 94%. The data in Table 5 demonstrate that formulations 3, 5, and 6 represented the best preservation of lyophilized PBMCs via a Trypan Blue Staining Assay.

In particular, incubating buffer with an increased polysucrose percentage and DMSO content resulted in the highest % of cells that retained their viability after lyophilization and rehydration, as assessed by Trypan Blue exclusion (formulation 3). The exclusion of Trypan Blue stain (clear cells) represents viable cells, since viable cells will exclude the Trypan Blue dye (e.g., a viable cell possesses an intact cell membrane), whereas dead cells are permeable to the dye (blue cells).

Formulation 5 included trehalose, a cryoprotectant, and BSA which may also act as a preservative, in generic PBS buffer. The addition of trehalose and BSA to PBS buffer alone (e.g., not including polysucrose) produced lyophilized PBMCs that retained their ability to exclude Trypan Blue upon rehydration (formulation 5) (Table 5).

The data also show that using RPMI-1640 (cell media) as the base buffer with the addition of trehalose, sorbitol, BSA, and polysucrose (formulation 6) preserved lyophilized PBMCs ability to exclude Trypan Blue more effectively than any formulation with the exception of formulation 3. Formulation 6 also showed the best preservation of lyophilized PBMC MTT activity of any tested lyophilized sample, as discussed more fully below.

PBMC viability and/or activity was also tested via the MTT Assay, a colorimetric assay measuring absorbance for assessing cell metabolic activity (Stockert, J. C., Tetrazolium salts and formazan products in cell biology: viability assessment, fluorescence imaging, and labeling perspectives, Acta Histochemica, 120(3), 159-167 (2018)). The MTT assay is a direct measurement of oxidoreductase enzymatic activity which is an indicator of cell viability/metabolic activity and proliferation.

The MTT Assay was performed according to the following protocol:
a. Fresh PBMCs were supplied at 300,000,000 cells in 1.5 mL PBS with 5% (fetal bovine serum) FBS and 0.5% BSA to a concentration of about 200K/μL PBMCs.
b. The fresh PBMCs were incubated separately in each of the described formulations above (e.g., formulations 1-6) for 30 minutes at 37° C. and subsequently lyophilized.
c. Lyophilized PBMC formulations, as described above, were rehydrated in 1 mL of cell culture water for 10 minutes and the MTT assay was performed in the rehydrated buffer. All reactions were conducted in Eppendorf tubes.
d. The input cell concentrations per assay was based on a presumed cell count prior to lyophilization (˜500K)
e. The MTT assay was performed with the abcam MTT assay kit (ab211091) (Cambridge, UK, www.abcam.com, www.abcam.com/mtt-assay-kit-cell-proliferation-ab211091) according to the manufacturer's instructions, with the alteration that UV absorbance was measured at 590 nm instead of 570 nm.

FIG. 1 shows the photographic results of fresh PBMCs in the MTT assay. The first Eppendorf tube (leftmost) is a negative control with only RPMI media. The 500K (“K” is a unit of measurement referring to thousands (K) of cells) RPMI sample shows purple coloration (appears as dark grey in the black-white image) indicative of cells' present in the sample ability to grow and divide. Formulation 6 sample is formulation 6 (also referred to as buffer 6 in FIG. 1) alone (control). The photographic results also show that formulation 6 enhanced (e.g., enhanced growth potential) the % max activity of fresh PBMCs, as measured by an MTT assay, relative to RPMI media alone.

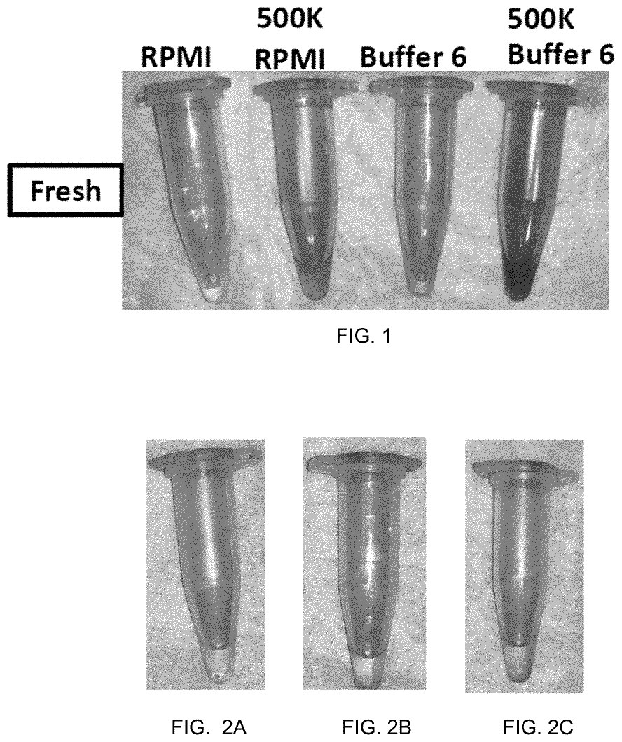

FIGS. 2A-C show the photographic results of rehydrated lyophilized PBMCs in the MTT assay. FIGS. 2A-C correspond to formulations 3, 5, and 6, respectively. While little color (showing as lighter gray in grayscale photo) was observed in formulation 3 (FIG. 2A) or formulation 5 (FIG. 2B), color (showing as darker gray in grayscale photo and reflected in the tip of the Eppendorf tube) was observed in formulation 6, suggesting that formulation 6 enhanced the growth potential of PBMCs ability as measured by the MTT assay (e.g., % max activity).

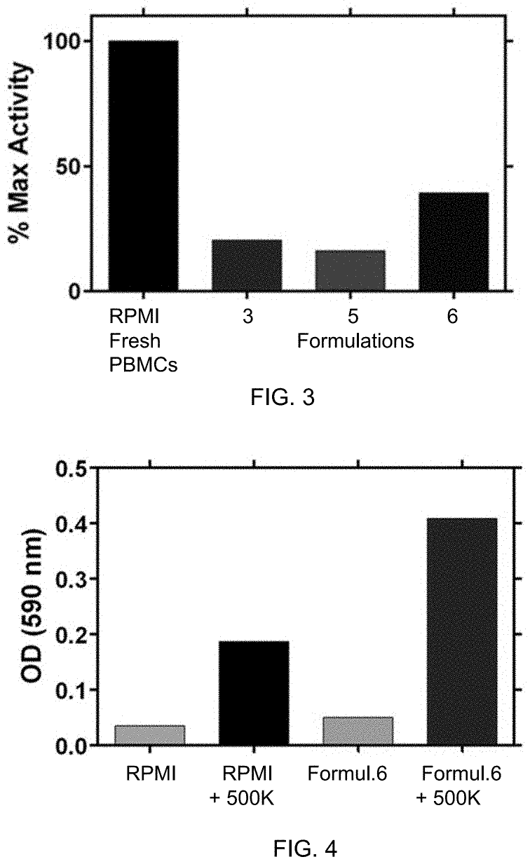

FIG. 3 shows a graphical representation of MTT assay results. The graph shows % Max Activity of the lyophilized PBMCs preserved in formulation 3, formulation 5, and formulation 6 (shown as buffer 3, 5, and 6), relative to the RPMI fresh PBMC sample (e.g., set to 100%). Formulation 6 showed the highest % Max Activity of lyophilized PBMCs, indicating formulation 6 provided the best preservation of lyophilized PBMCs as measured by MTT assay.

FIG. 4 shows a graphical representation of MTT assay results. The graph shows the optical density (OD) measured at 590 nm for fresh PBMCs incubated in either RPMI media or formulation 6 (shown as buffer 6) that had been used to rehydrate lyophilized PBMCs. RPMI and formulation 6 controls are shown in columns 1 and 3, respectively. PBMCs (500K) were contacted with either RPMI (column 2) or with formulation 6 that had been used to rehydrate lyophilized PBMCs (column 4). Formulation 6 was isolated by rehydrating lyophilized PBMCs, centrifuging the sample, and filtering as described above.

Formulation buffer 6 showed enhanced viability/metabolic activity in fresh PBMCs. Formulation buffer 3 resulted in a higher viability percentage than buffer formulation 6. While not wishing to be limited by any theory, the increased viability may be due to either the DMSO and/or the increased percentage of polysucrose. The Trypan Blue assay measures a cell's ability to exclude dye (e.g., maintain a stable lipid bilayer), whereas the MTT assay measures intracellular metabolic activity.

The MTT assay is associated with a living cell's ability to metabolize, grow and divide and is indicative of cellular metabolism, proliferation and living cells. As shown in FIG. 4 fresh PMBCs incubated in formulation 6 more than doubled the metabolic/proliferative activity of fresh PBMCs, suggesting a potential new cell media and/or rehydration formulation for preserved and/or fresh cells, including PBMCs. During the lyophilization process some PBMCs are ruptured thus releasing their cell contents. Without wishing to be bound by any theory, it is likely that proteins such as growth factors, chemokines, etc. from the cell lysate are present in the formulation 6 used to rehydrate lyophilized PBMCs and may account for the increase in MTT activity relative to PBMCs that have not been frozen and are suspended in RPMI media.

It is to be understood that while the methods and compositions have been described in conjunction with the detailed description thereof, the forgoing description is intended to illustrate and not limit the scope of the methods and compositions described herein, which is defined by the scope of the appended claims. Other aspects, advantages, and modifications are within the scope of the following claims.

1-4. (canceled)

5. A method of preparing freeze-dried peripheral blood mononuclear cells (PBMCs), the method comprising:
A) incubating PBMCs at a temperature of between 35° C. and 40° C. for between 10 minutes and 120 minutes, in an aqueous mixture to form incubated PBMCs suspended in the aqueous mixture, wherein the aqueous mixture comprises;
i) trehalose, wherein the trehalose is present in the aqueous mixture at a concentration of from 0.1% (w/v) to 10.0% (w/v);
ii) an excipient comprising polysucrose and/or albumin, wherein the polysucrose and/or the albumin are present in the aqueous mixture, at a concentration individually or in combination from 4.0% (w/v) to 20.0% (w/v);
iii) a buffer; and
iv) either one or both sorbitol and PBMC cell culture media components, wherein the sorbitol if present, is present in the aqueous mixture at a concentration from 0.1% (w/v) to 5.0% (w/v), and the cell culture media components if present comprise amino acids, vitamins, and inorganic salts, at effective concentrations to facilitate culturing of the PBMCs; and
B) lyophilizing the incubated PBMCs suspended in the aqueous mixture to form a solid composition comprising the freeze-dried PBMCs.

6. The method of claim 5, wherein the aqueous mixture comprises the PBMC cell culture media components at concentrations that effectively support PBMC cell culturing, and the buffer comprises the buffer in the cell culture media.

7-8. (canceled)

9. The method of claim 6, wherein the PBMCs are never suspended in a liquid comprising DMSO during the method.

10. The method of claim 9, wherein between 1% and 10% of the freeze-dried PBMCs are viable in the solid composition comprising freeze-dried PBMCs.

11-20. (canceled)

21. The method of claim 6, wherein the solid composition, or the aqueous mixture Noes not comprise fibrinogen.

22. The method of claim 6, wherein the method does not comprise heating the solid composition comprising freeze-dried PBMCs to a temperature of 60° C. or above for more than 1 hour.

23. The method of claim 6, wherein the incubating is performed for between 15 min and 60 min at about 37° C.

24. The method of claim 6, wherein the trehalose is present in the aqueous mixture at a concentration of from 2.0% (w/v) to 6.0% (w/v).

25. The method of claim 6, wherein the aqueous mixture comprises sorbitol at a concentration from about 0.1% (w/v) to about 5.0% (w/v).

26. The method of claim 25, wherein the aqueous mixture comprises albumin at a concentration from 3.0% (w/v) to 7.0% (w/v).

27. The method of claim 25, wherein the aqueous mixture comprises polysucrose at a concentration from 3.0% (w/v) to 7.0% (w/v).

28. The method of claim 25, wherein trehalose is present in the aqueous mixture at a concentration of from 2.0% (w/v) to 6.0% (w/v) and polysucrose is present at a concentration from 3.0% (w/v) to 7.0% (w/v).

29. (canceled)

30. The method of claim 28, wherein the freeze-dried PBMCs have enhanced metabolic activity compared to freeze-dried PBMCs lyophilized in a medium comprising 2% to 3% DMSO.

31-33. (canceled)

34. The method of claim 28, wherein the aqueous mixture comprises RPMI-1640 cell culture media.

35-36. (canceled)

37. The method of claim 6, wherein the aqueous mixture comprises the PBMC cell culture media components at concentrations that effectively support PBMC cell culturing, wherein trehalose is present in the aqueous mixture at a concentration of from 2.0% (w/v) to 6.0% (w/v) and polysucrose is present at a concentration from 3.0% (w/v) to 7.0% (w/v).

38. (canceled)

39. The method of claim 10, wherein the freeze-dried PBMCs have enhanced metabolic activity compared to the freeze-dried PBMCs lyophilized in a medium comprising 2% to 3% DMSO.

40-42. (canceled)

43. A peripheral blood mononuclear cell (PBMC) composition in the form of a solid, comprising:
A) 10-20% (w/v) trehalose;
B) 50-70% (w/v) polysucrose and/or albumin;
C) 10-20% DMSO; and
D) a population of between 1×10⁵ and 1×10¹¹ freeze-dried peripheral blood mononuclear cells (PBMCs),
wherein at least 1% of the PBMCs in said population, when rehydrated are viable.

44. The PBMC composition of claim 43, wherein the composition comprises between 14-18% trehalose, between 12-18% DMSO, and between 60 and 70% polysucrose, and wherein between 15% and 25% of the freeze-dried PBMCs are viable.

45. The PBMC composition of claim 43, wherein the composition comprises between 14-18% trehalose, between 12-18% DMSO, between 60 and 70% polysucrose, and wherein between 15% and 20% of the freeze-dried PBMCs are viable.

46-47. (canceled)

48. The method of claim 6, wherein the solid composition comprising freeze-dried PBMCs has a property that between 1 and 25% of the PBMCs have metabolic activity and are viable when rehydrated.
